(19) United States
(12) Patent Application Publication
(10) Pub. No.: US 2026/0207776 A1
(43) Pub. Date: Jul. 23, 2026

(54) IN VIVO AND EX VIVO PRODUCTION OF CAR-NEUTROPHILS AND USE THEREOF TO TREAT AND IMAGE CANCER

(71) Applicants: Purdue Research Foundation, West Lafayette, IN (US); The Penn State Research Foundation, University Park, PA (US)

(72) Inventors: Xiaoping BAO, West Lafayette, IN (US); Yun CHANG, West Lafayette, IN (US); Qing DENG, West Lafayette, IN (US); Xiaojun LIAN, State College, PA (US)

(21) Appl. No.: 19/139,761

(22) PCT Filed: Dec. 18, 2023
(86) PCT No.: PCT/US2023/084694

§ 371 Date: Jun. 16, 2025

Related U.S. Application Data
(60) Provisional application No. 63/433,299, filed on Dec. 16, 2022.

(51) Int. Cl. A61K 48/00; A61K 38/16; A61K 39/395; A61K 40/10; A61K 40/31; A61K 40/42; A61P 35/00; C07K 14/00; C07K 16/30; C12N 5/0787

(52) U.S. Cl. CPC A61K 48/005; A61K 38/16; A61K 39/3955; A61K 40/10; A61K 40/31; A61K 40/4276; A61K 48/0033; A61K 48/0083; A61P 35/00; C07K 14/00; C07K 16/3069; C12N 5/0642; C12N 2510/00

(57) ABSTRACT

Modified RNA (modRNA) constructs are provided for use in combination to produce chimeric antigen receptor (CAR)-expressing neutrophils. In vivo and ex vivo methods of producing CAR-expressing neutrophils are also provided, as are uses of such modRNA constructs in the preparation of a medicament.

### PRIORITY

This application is related to and claims the priority benefit of U.S. Provisional Patent Application No. 63/433,299 filed Dec. 16, 2022. The content of the aforementioned application is hereby incorporated by reference in its entireties into this disclosure.

### TECHNICAL FIELD

The present disclosure relates to chimeric antigen receptor (CAR)-neutrophils, synthetic modified mRNA (modRNA), and the treatment and imaging of cancer, such as brain cancer, e.g., glioblastoma, and prostate cancer.

### STATEMENT OF SEQUENCE LISTING

A computer-readable form (CRF) of the Sequence Listing is submitted concurrently with this application, which is herein incorporated by reference in its entirety. The content of the computer-readable form is identical to the written sequence listing set forth herein.

SEQ ID NO: 1 is a first nucleic acid sequence that encodes a k-turn:

GGGCGTGATCCGAAAGGTGACCC.

SEQ ID NO: 2 is a second nucleic acid sequence that encodes a k-turn:

GGGCGTGATGCGAAAGCTGACCC.

SEQ ID NO: 3 is a nucleic acid sequence that encodes a microRNA223 (miRNA-223) binding site: TGGGGTATTTGACAAACTGACA.

SEQ ID NO: 4 is a nucleic acid sequence that encodes a miRNA-223 binding site with a bulge:

TGGGGTATTTctgAACTGACA.

SEQ ID NO: 5 is a nucleic acid sequence that encodes a microRNA-142-5p (miRNA-142-5p) binding site: AGTAGTGCTTTCTACTTTATGGG.

SEQ ID NO: 6 is a nucleic acid sequence that encodes a miRNA-142-5p binding site with a bulge-1: AGTAGTGCTTTggaTTTATGGG.

SEQ ID NO: 7 is a nucleic acid sequence that encodes a miRNA-142-5p binding site with a bulge-2: AGTAGTGCTagaACTTTATGGG.

SEQ ID NO: 8 is an amino acid sequence for a L7Ae-eGFP: k-turn:
MYVRFEVPEDMQNEALSLLEKVRESGKVKKGTNETTKAVERGLAKLVYIAEDVDPPEI
VAHLPLLCEEKNVPYIYVKSKNDLGRAVGIEVPCASAAIINEGELRKELGSLVEKIKGLQ
KGSGATNFSLLKQAGDVEENPGPMVSKGEELFTGVVPILVELDGDVNGHKFSVSGEGE
GDATYGKLTLKFICTTGKLPVPWPTLVTTLTYGVQCFSRYPDHMKQHDFFKSAMPEGY
VQERTIFFKDDGNYKTRAEVKFEGDTLVNRIELKGIDFKEDGNILGHKLEYNYNSHNVY
IMADKQKNGIKVNFKIRHNIEDGSVQLADHYQQNTPIGDGPVLLPDNHYLSTQSALSKD
PNEKRDHMVLLEFVTAAGITLGMDELYKPKKKRKV.
SEQ ID NO: 9 is an amino acid sequence for a 2x k-turn tdTomato:
MVSKGEEVIKEFMRFKVRMEGSMNGHEFEIEGEGEGRPYEGTQTAKLKVTKGGPLPFA
WDILSPQFMYGSKAYVKHPADIPDYKKLSFPEGFKWERVMNFEDGGLVTVTQDSSLQD
GTLIYKVKMRGTNFPPDGPVMQKKTMGWEASTERLYPRDGVLKGEIHQALKLKDGGH
YLVEFKTIYMAKKPVQLPGYYYVDTKLDITSHNEDYTIVEQYERSEGRHHLFLGHGTGS
TGSGSSGTASSEDNNMAVIKEFMRFKVRMEGSMNGHEFEIEGEGEGRPYEGTQTAKLK
VTKGGPLPFAWDILSPQFMYGSKAYVKHPADIPDYKKLSFPEGFKWERVMNFEDGGLV
TVTQDSSLQDGTLIYKVKMRGTNFPPDGPVMQKKTMGWEASTERLYPRDGVLKGEIH
QALKLKDGGHYLVEFKTIYMAKKPVQLPGYYYVDTKLDITSHNEDYTIVEQYERSEGR
HHLFLYGMDELYK.
SEQ ID NO: 10 is an amino acid sequence for a 2x k-turn CLTX human 4-1BB CD3z
chimeric antigen receptor (CAR):
MLLLVTSLLLCELPHPAFLLIPMCMPCFTTDHQMARKCDDCCGGKGRGKCYGPQCLCR
ESKYGPPCPPCPAPEFLGGPSVFLFPPKPKDTLMISRTPEVTCVVVDVSQEDPEVQFNWY
VDGVEVHNAKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQPENNYKTTP
PVLDSDGSFFLYSRLTVDKSRWQEGNVFSCSVMHEALHNHYTQKSLSLSLGKIYIWAPL
AGTCGVLLLSLVITLYCNHRNRFSVVKRGRKKLLYIFKQPFMRPVQTTQEEDGCSCRFP
EEEEGGCELRVKFSRSADAPAYQQGQNQLYNELNLGRREEYDVLDKRRGRDPEMGGK
PRRKNPQEGLYNELQKDKMAEAYSEIGMKGERRRGKGHDGLYQGLSTATKDTYDALH
MQALPPR.
SEQ ID NO: 11 is an amino acid sequence for a 2x k-turn CLTX mouse CD28 CD3z
CAR:
MLLLVTSLLLCELPHPAFLLIPMCMPCFTTDHQMARKCDDCCGGKGRGKCYGPQCLCR
ESKYGPPCPPCPAPEFLGGPSVFLFPPKPKDTLMISRTPEVTCVVVDVSQEDPEVQFNWY
VDGVEVHNAKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQPENNYKTTP
PVLDSDGSFFLYSRLTVDKSRWQEGNVFSCSVMHEALHNHYTQKSLSLSLGKFWALVV
VAGVLFCYGLLVTVALCVIWTNSRRNRLLQSDYMNMTPRRPGLTRKPYQPYAPARDF
AAYRPRAKFSRSAETAANLQDPNQLYNELNLGRREEYDVLEKKRARDPEMGGKQQRR
RNPQEGVYNALQKDKMAEAYSEIGTKGERRRGKGHDGLYQGLSTATKDTYDALHMQ
TLAPR.
SEQ ID NO: 12 is an amino acid sequence for a CLTX canine CD28 CD3z CAR:
MLLLVTSLLLCELPHPAFLLIPMCMPCFTTDHQMARKCDDCCGGKGRGKCYGPQCLCR
ESKYGPPCPPCPAPEFLGGPSVFLFPPKPKDTLMISRTPEVTCVVVDVSQEDPEVQFNWY
VDGVEVHNAKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQPENNYKTTP
PVLDSDGSFFLYSRLTVDKSRWQEGNVFSCSVMHEALHNHYTQKSLSLSLGKFWALVV
VGAVLVFYSLLVTVALCAYWIKSKSSRILQSDYMNMTPRRPGPTRRHYQPYAPARDFA
AYRSLRAKFGRSAAAPEHQQGPNQLYNELNLRGREEYEVLDKRRGLDPEMGGKQRKR
NPQEVVYNALOKDKMAEAYSEIGIKSENQRRRGKGHDGLYQGLSTATKDTYDALHMQ
ALPPR.
SEQ ID NO: 13 is an amino acid sequence for a peptide that targets mouse neutrophils:
LQIQSWSSSP.
SEQ ID NO: 14 is an amino acid sequence for a peptide that targets human neutrophils:
KFPDLDSRRLPHMSL.

### BACKGROUND

Glioblastoma (GBM) is one of the most aggressive and lethal solid tumors in humans. While efficacious therapeutics, such as emerging chimeric antigen receptor (CAR)-T cells and chemotherapeutics, have been developed to treat various cancers, their effectiveness in GBM treatment has been hindered largely by the blood-brain barrier and blood-brain-tumor barriers.

Human neutrophils can effectively cross physiological barriers and display effector immunity against pathogens; however, the short lifespan and resistance to genome editing of primary neutrophils have limited their broad application in immunotherapy. CAR-neutrophils have been engineered from human pluripotent stem cells (hPSCs) de novo, but in vitro cell engineering can be burdensome and time-consuming.

In view of the above, it is an object of the present disclosure to provide a method of producing CAR-neutrophils in vivo and ex vivo. This and other objects and advantages, as well as inventive features, will be apparent from the detailed description provided herein.

### SUMMARY

A method of producing chimeric antigen receptor (CAR)-expressing neutrophils in a subject is provided. The method comprises administering to the subject modified RNA (modRNA) comprising (i) a modRNA construct comprising (a) a nucleotide sequence encoding the archaeal ribosomal protein L7Ae and (b) at least one neutrophil-specific microRNA recognition sequence, wherein the at least one neutrophil-specific microRNA recognition sequence is downstream of the nucleotide sequence encoding L7Ae and (ii) a modRNA construct comprising (a) a nucleotide sequence encoding a CAR and (b) at least one k-turn, wherein the at least one k-turn is upstream of the nucleotide sequence encoding CAR. The modRNA is endocytosed by the neutrophils in the subject, and the neutrophil expresses the CAR. The subject can be a human, and the modRNA construct of (i) can comprise at least one neutrophil-specific microRNA recognition sequence selected from miR-233, miR-142, and a combination thereof. The subject can be a human, and the CAR can comprise a human CD4 transmembrane domain and a human CD3ζ intracellular domain. The subject can be a human, and the CAR can comprise a human CD8 transmembrane domain, a human 41BB co-stimulatory domain, and a human CD3ζ intracellular domain. The CAR can comprise a 36-amino acid glioblastoma (GBM)-targeting chlorotoxin peptide or other brain tumor-targeting ligand (e.g., IL-13) or a single-chain variable fragment (scFv). The CAR can comprise a ligand that targets prostate-specific membrane antigen (PSMA). The modRNA constructs can be administered in an exosome. The exosome can be administered systemically. The modRNA can be administered in a lipid nanoparticle (LNP). The LNP can be administered systemically. The method can further comprise administering a nanodrug. The nanodrug can be a chemotherapeutic agent. The subject can have cancer. The cancer can be a brain cancer. The brain cancer can be glioblastoma. The cancer can be prostate cancer.

A method of producing CAR-expressing neutrophils ex vivo for administration to a subject is also provided. The method comprises (i) isolating neutrophils from the subject, (ii) contacting the isolated neutrophils with two modified RNAs (modRNAs) comprising (a) a modRNA construct comprising (i′) a nucleotide sequence encoding the archaeal ribosomal protein L7Ae and (ii′) at least one neutrophil-specific microRNA recognition sequence, wherein the at least one neutrophil-specific microRNA recognition sequence is downstream of the nucleotide sequence encoding L7Ae and (b) a modRNA construct comprising (i′) a nucleotide sequence encoding a CAR and (ii) at least one k-turn, wherein the at least one k-turn is upstream of the nucleotide sequence encoding CAR, whereupon the modRNA is endocytosed by the neutrophils, and the neutrophils express the CAR, and (iii) administering the neutrophils to the subject. The subject can be a human, and the modRNA construct of (a) can comprise at least one neutrophil-specific microRNA recognition sequence selected from miR-233, miR-142, and a combination thereof. The subject can be a human, and the CAR can comprise a human CD4 transmembrane domain and a human CD3ζ intracellular domain. The subject can be a human, and the CAR can comprise a human CD8 transmembrane domain, a human 41BB co-stimulatory domain, and a human CD3ζ intracellular domain. The CAR can comprise a 36-amino acid GBM-targeting chlorotoxin peptide or other brain tumor-targeting ligand (e.g., IL-13) or a scFv. The CAR can comprise a ligand that targets PSMA. The method can further comprise administering to the subject a nanodrug. The nanodrug can be a chemotherapeutic agent. The subject can have cancer. The cancer can be a brain cancer. The brain cancer can be glioblastoma. The cancer can be prostate cancer.

A composition is also provided. The composition comprises (i) a modRNA construct comprising (a) a nucleotide sequence encoding the archaeal ribosomal protein L7Ae and (b) at least one neutrophil-specific microRNA recognition sequence, wherein the at least one neutrophil-specific microRNA recognition sequence is downstream of the nucleotide sequence encoding L7Ae and (ii) a modRNA construct comprising (a) a nucleotide sequence encoding a CAR and (b) at least one k-turn, wherein the at least one k-turn is upstream of the nucleotide sequence encoding CAR. The modRNA construct of (i) can comprise at least one neutrophil-specific microRNA recognition sequence selected from miR233, miR142, and a combination thereof. The CAR can comprise a human CD4 transmembrane domain and a human CD3ζ intracellular domain. The CAR can comprise a human CD8 transmembrane domain, a human 41BB co-stimulatory domain, and a human CD3ζ intracellular domain. The CAR can comprise a 36-amino acid GBM-targeting chlorotoxin peptide or other brain tumor-targeting ligand (e.g., IL-13) or a scFv. The CAR can comprise a ligand that targets PSMA.

Further provided is an exosome. The exosome comprises an above-described composition.

Still further provided is an LNP. The LNP comprises an above-described composition.

Additional methods of producing CAR-expressing neutrophils in a subject are provided. In certain embodiments, the method of producing CAR-expressing neutrophils in a subject comprises administering to the subject at least one modRNA construct comprising a first modRNA construct comprising a nucleotide sequence encoding a CAR and at least one k-turn, wherein the at least one k-turn is upstream of the nucleotide sequence encoding CAR, whereupon when the at least one modRNA construct is endocytosed by a neutrophil in the subject, and the neutrophil expresses the CAR.

The at least one modified construct can comprise the first modRNA construct and a second modRNA construct comprising a nucleotide sequence encoding an L7Ae and at least one neutrophil-specific miRNA recognition sequence, wherein the at least one neutrophil-specific miRNA recognition sequence is downstream of the nucleotide sequence encoding L7Ae. The neutrophil-specific miRNA can be or comprise miR-233, miR-142, or both miR-233 and miR-142.

The subject can be a mammal. The subject can be a canine. The subject can be human. The CAR encoded by the first modRNA construct (e.g., the modRNA CAR construct) can comprise a neutrophil-specific transmembrane domain. The neutrophil-specific transmembrane domain can be or comprise a toll-like receptor (TLR) 4 polypeptide, a TLR2 polypeptide, a MET polypeptide, a granulocyte colony stimulating factor receptor (G-CSFR), a Myd88 polypeptide, a TRIF polypeptide, a Syk peptide, a CD40 polypeptide, CD32a, Dectin-1, a IL-6 receptor (IL6R), an Fc Epsilon Receptor Ig (FCER1G) polypeptide, a TLR7, or a CD16 transmembrane polypeptide, a CD8 polypeptide, a CD28 polypeptide, an OX40 polypeptide, an ICOS polypeptide, a CTLA-4 polypeptide, a PD-1 polypeptide, a LAG-3 polypeptide, a BTLA polypeptide, a natural killer group 2D (NKG2D), Dectin-1, CD16, or other Fcγ receptors. The CAR can comprise a neutrophil-specific transmembrane and/or co-stimulatory domain, wherein: the neutrophil-specific co-stimulatory domain can optionally be or comprise a 41BB co-stimulatory domain or a CD28 polypeptide; and the neutrophil-specific transmembrane domain can optionally be or comprise CD32a, CD4, NKG2D, Dectin-1, a CD3ζ polypeptide, an IL-6 receptor, or CD16.

The subject can be human and the CAR can comprise a human CD4 transmembrane domain and a human CD3ζ intracellular domain. The subject can be human and the CAR can comprise a human CD8 transmembrane domain, a human 41BB co-stimulatory domain, and a human CD3ζ intracellular domain. The CAR can comprise a 36-amino acid glioblastoma (GBM)-targeting chlorotoxin peptide or other brain-targeting ligand, a tumor-targeting ligand, or a single-chain variable fragment (scFv). The CAR can comprise a ligand that targets prostate-specific membrane antigen (PSMA).

The modRNA construct(s) can be administered in an exosome to circulating neutrophils in vivo. The modRNA construct(s) can be administered systemically to the subject in a carrier to circulating neutrophils in vivo. The modRNA construct(s) can be administered systemically to the subject in an exosome to circulating neutrophils in vivo. The modRNA construct(s) can be administered in a lipid nanoparticle (LNP). The modRNA construct(s) can be administered systemically to the subject in a LNP.

In certain embodiments, the method further comprises administering a therapeutically effective amount of a drug, optionally a nanodrug and/or a prodrug, to the subject. The method cn further comprise administering a second therapy to the subject. The method can further comprise administering a second therapy to the subject, wherein the second therapy comprises surgical removal of one or more cancerous cells from the subject, chemotherapy, imaging, and/or radiotherapy. The method can further comprise administering a therapeutically effective amount of a nanodrug to the subject, wherein the nanodrug is a chemotherapeutic agent.

The subject can have cancer. The subject can have brain cancer. The brain cancer can be glioblastoma. The subject can have prostate cancer.

The neutrophil that endocytosis the at least one modRNA construct can be an isolated neutrophil and the method can further comprise administering the isolated neutrophil that endocytosed the at least one modRNA construct to the subject. The method can further comprise isolating one or more neutrophils from the subject.

The at least one neutrophil-specific microRNA recognition sequence of the second modRNA construct can be or comprise SEQ ID NO: 3, SEQ ID NO: 4, SEQ ID NO: 5, SEQ ID NO: 6, SEQ ID NO: 7, or a functional variant of SEQ ID NO: 3, SEQ ID NO: 4, SEQ ID NO: 5, SEQ ID NO: 6, or SEQ ID NO: 7. The first modRNA construct can comprise SEQ ID NO: 8, SEQ ID NO: 9, or a functional variant of SEQ ID NO: 8 or SEQ ID NO: 9. The modRNA construct can comprise SEQ ID NO: 10, SEQ ID NO: 11, SEQ ID NO: 12, or a functional variant of SEQ ID NO: 10, SEQ ID NO: 11, or SEQ ID NO: 12. The first modRNA construct can comprise SEQ ID NO: 1, SEQ ID NO: 2, or a functional variant of SEQ ID NO: 1 or SEQ ID NO: 2.

In certain embodiments, the first modRNA construct comprises: a first sequence comprising SEQ ID NO: 1, SEQ ID NO: 2, or a functional variant of SEQ ID NO: 1 or SEQ ID NO: 2; and a second sequence comprising SEQ ID NO: 10, SEQ ID NO: 11, SEQ ID NO: 12, or a functional variant of SEQ ID NO: 10, SEQ ID NO: 11, or SEQ ID NO: 12; wherein the first sequence is upstream of the second sequence.

Methods of producing CAR-expressing neutrophils ex vivo for administration to a subject are also provided, in certain embodiments, such methods comprising: (i) isolating neutrophils from a subject, (ii) contacting the isolated neutrophils with at least one modRNA construct, the at least one modRNA construct comprising a first modRNA construct comprising a nucleotide sequence encoding a CAR and at least one k-turn, wherein the at least one k-turn is upstream of the nucleotide sequence encoding CAR; whereupon when the modRNA is endocytosed by the isolated neutrophils, the isolated neutrophils express the CAR, and (iii) administering the CAR-expressing neutrophils to the subject.

The at least one modRNA construct can comprise the first modRNA construct and a second modRNA construct, the second modRNA construct comprising a nucleotide sequence encoding an L7Ae and at least one neutrophil-specific miRNA recognition sequence, wherein the at least one neutrophil-specific miRNA recognition sequence is downstream of the nucleotide sequence encoding L7Ae. The subject can be human and the at least one neutrophil-specific miRNA recognition sequence of the second modRNA construct can be or comprise miR-233, miR-142, or both miR-233 and miR-142.

The subject can be human and the CAR can comprise a human CD4 transmembrane domain and a human CD3ζ intracellular domain. The subject can be human and the CAR can comprise a human CD8 transmembrane domain, a human 41BB co-stimulatory domain, and a human CD3ζ intracellular domain. The CAR (i.e., encoded by the first modRNA construct) can comprise a 36-amino acid GBM-targeting chlorotoxin peptide or other brain tumor-targeting ligand. The CAR can comprise a ligand that targets PSMA. The CAR can comprise a neutrophil-specific transmembrane domain. The CAR can comprise a neutrophil-specific transmembrane domain is or comprises a TLR4 polypeptide, a TLR2 polypeptide, a MET polypeptide, a G-CSFR, a Myd88 polypeptide, a TRIF polypeptide, a Syk peptide, a CD40 polypeptide, CD32a, Dectin-1, a IL6R, an FCER1G polypeptide, a TLR7, or a CD16 transmembrane polypeptide, a CD8 polypeptide, a CD28 polypeptide, an OX40 polypeptide, an ICOS polypeptide, a CTLA-4 polypeptide, a PD-1 polypeptide, a LAG-3 polypeptide, a BTLA polypeptide, a NKG2D, Dectin-1, CD16, or other Fcγ receptors. In certain embodiments, the CAR comprises a neutrophil-specific transmembrane and/or co-stimulatory domain, wherein: the neutrophil-specific co-stimulatory domain can optionally be or comprise a 41BB co-stimulatory domain or a CD28 polypeptide; and the neutrophil-specific transmembrane domain can optionally be or comprise CD32a, CD4, NKG2D, Dectin-1, a CD3ζ polypeptide, an IL-6 receptor, or CD16.

The method can further comprise administering to the subject a therapeutically effective amount of a drug and, optionally, the drug comprising a nanodrug or a prodrug. In certain embodiments, the method comprises administering to the subject a therapeutically effective amount of a chemotherapeutic agent.

The subject can have cancer. The subject can have brain cancer. The brain cancer can be glioblastoma. The subject can have prostate cancer.

The at least one neutrophil-specific microRNA recognition sequence of the second modRNA construct can be or comprise SEQ ID NO: 3, SEQ ID NO: 4, SEQ ID NO: 5, SEQ ID NO: 6, SEQ ID NO: 7, or a functional variant of SEQ ID NO: 3, SEQ ID NO: 4, SEQ ID NO: 5, SEQ ID NO: 6, or SEQ ID NO: 7. The first modRNA construct can comprise SEQ ID NO: 8, SEQ ID NO: 9, or a functional variant of SEQ ID NO: 8 or SEQ ID NO: 9. The first modRNA construct can comprise SEQ ID NO: 10, SEQ ID NO: 11, SEQ ID NO: 12, or a functional variant of SEQ ID NO: 10, SEQ ID NO: 11, or SEQ ID NO: 12. The first modRNA construct can comprise SEQ ID NO: 1, SEQ ID NO: 2, or a functional variant of SEQ ID NO: 1 or SEQ ID NO: 2. The first modRNA construct can comprise: a first sequence comprising SEQ ID NO: 1, SEQ ID NO: 2, or a functional variant of SEQ ID NO: 1 or SEQ ID NO: 2; and a second sequence comprising SEQ ID NO: 10, SEQ ID NO: 11, SEQ ID NO: 12, or a functional variant of SEQ ID NO: 10, SEQ ID NO: 11, or SEQ ID NO: 12; wherein the first sequence is upstream of the second sequence.

Compositions are also provided. In certain embodiments, the composition comprises a first modRNA construct comprising a nucleotide sequence encoding a CAR and at least one k-turn, wherein the at least one k-turn is upstream of the nucleotide sequence encoding the CAR. The composition can further comprise a second modRNA construct comprising a nucleotide sequence encoding the L7Ae and at least one neutrophil-specific miRNA recognition sequence, wherein the at least one neutrophil-specific miRNA recognition sequence is downstream of the nucleotide sequence encoding L7Ae. In certain embodiments, the at least one neutrophil-specific miRNA recognition sequence of the second modRNA construct is or comprises miR-233, miR-142, or both miR-233 and miR-142.

In certain embodiments of the composition, the CAR encoded by the first modRNA construct comprises a human CD4 transmembrane domain and a human CD3ζ intracellular domain. The CAR can comprise a human CD8 transmembrane domain, a human 41BB co-stimulatory domain, and a human CD3ζ intracellular domain. The CAR can comprise a 36-amino acid GBM-targeting chlorotoxin peptide or other brain tumor-targeting ligand (e.g., IL-13) or a scFv. The CAR can comprise a ligand that targets PSMA.

In certain embodiments, the at least one neutrophil-specific miRNA recognition sequence of the second modRNA construct is or comprises SEQ ID NO: 3, SEQ ID NO: 4, SEQ ID NO: 5, SEQ ID NO: 6, SEQ ID NO: 7, or a functional variant of SEQ ID NO: 3, SEQ ID NO: 4, SEQ ID NO: 5, SEQ ID NO: 6, or SEQ ID NO: 7. In certain embodiments, the first modRNA construct comprises SEQ ID NO: 8, SEQ ID NO: 9, or a functional variant of SEQ ID NO: 8 or SEQ ID NO: 9. In certain embodiments, the first modRNA construct comprises SEQ ID NO: 10, SEQ ID NO: 11, SEQ ID NO: 12, or a functional variant of SEQ ID NO: 10, SEQ ID NO: 11, or SEQ ID NO: 12. In certain embodiments, the first modRNA construct comprises SEQ ID NO: 1, SEQ ID NO: 2, or a functional variant of SEQ ID NO: 1 or SEQ ID NO: 2.

In certain embodiments of the composition, the first modRNA construct comprises: a first sequence comprising SEQ ID NO: 1, SEQ ID NO: 2, or a functional variant of SEQ ID NO: 1 or SEQ ID NO: 2; and a second sequence comprising SEQ ID NO: 10, SEQ ID NO: 11, SEQ ID NO: 12, or a functional variant of SEQ ID NO: 10, SEQ ID NO: 11, or SEQ ID NO: 12; wherein the first sequence is upstream of the second sequence.

The CAR can comprise a neutrophil-specific transmembrane domain. The CAR can comprise a neutrophil-specific transmembrane domain that is or comprises a TLR 4 polypeptide, a TLR2 polypeptide, a MET polypeptide, a G-CSFR, a Myd88 polypeptide, a TRIF polypeptide, a Syk peptide, a CD40 polypeptide, CD32a, Dectin-1, a IL-6R, an FCER1G polypeptide, a TLR7, or a CD16 transmembrane polypeptide, a CD8 polypeptide, a CD28 polypeptide, an OX40 polypeptide, an ICOS polypeptide, a CTLA-4 polypeptide, a PD-1 polypeptide, a LAG-3 polypeptide, a BTLA polypeptide, a NKG2D, Dectin-1, CD16, or other Fcγ receptors. The CAR can comprise a neutrophil-specific transmembrane and/or co-stimulatory domain, wherein: the neutrophil-specific co-stimulatory domain can optionally be or comprise a 41BB co-stimulatory domain or a CD28 polypeptide; and the neutrophil-specific transmembrane domain can optionally be or comprise CD32a, CD4, NKG2D, Dectin-1, a CD3 polypeptide, an IL-6 receptor, or CD16.

Additionally, embodiments of exosomes are provided that comprise any of the compositions hereof. NPs are also provided that comprise any of the compositions hereof. The NP can be a LNP.

Uses of at least one modRNA constructs in the preparation of a medicament for treating a cancer are provided, wherein, in certain embodiments, the at least one modRNA construct comprises a first modRNA construct comprising a nucleotide sequence encoding a CAR and at least one k-turn. The first modRNA can be any of the modRNA CAR constructs described herein.

In certain embodiments of the uses provided, the at least one modRNA constructs comprises a combination of the first modRNA construct and a second modRNA construct, the second modRNA construct comprising a nucleotide sequence encoding an L7Ae and at least one neutrophil-specific microRNA recognition sequence, wherein the at least one neutrophil-specific microRNA recognition sequence is downstream of the nucleotide sequence encoding L7Ae.

The cancer can be brain cancer. The brain cancer can be glioblastoma. The cancer can be prostate cancer.

In certain embodiments of the uses provided, the second modRNA construct comprises at least one neutrophil-specific microRNA recognition sequence that is or comprises miR-233, miR-142, or both miR-233 and miR-142. The CAR can comprise a human CD4 transmembrane domain and a human CD3ζ intracellular domain. The CAR can comprise a human CD8 transmembrane domain, a human 41BB co-stimulatory domain, and a human CD3ζ intracellular domain. The CAR can comprise a 36-amino acid GBM-targeting chlorotoxin peptide or other brain tumor-targeting ligand or a scFv. The CAR can comprise a ligand that targets PSMA.

In certain embodiments of the uses described herein, the at least one modRNA construct are encapsulated within a carrier. The medicament can be formulated for systemic administration. The at least one modRNA construct can be encapsulated within a NP, a microvesicle, or an exosome. In certain embodiments, the medicament is formulated for administration in combination with a therapeutically effective amount of a drug, optionally a nanodrug.

### BRIEF DESCRIPTION OF THE FIGURES

FIG. 1 is a schematic diagram of the method of the present disclosure, wherein (1) represents neutrophil specificity, (2) represents the endosome, (3) represents modRNA, (4) represents the ribosome, (5) represents CAR expression, (6) represents an immunological synapse, (7-1) represents phagocytosis by neutrophils, and (7-2) represents reactive oxygen species (ROS) and elastase transfer.

FIG. 2A shows a transmission electron microscopy (TEM) image of enhanced green fluorescent protein (eGFP) modified RNA (modRNA)-exosome. Scale bar=200 μm.

FIG. 2B shows flow cytometry analysis data of eGFP expression on various neutrophils treated by the indicated commercial lipid nanoparticle (LNP) or exosome modRNA delivery system, with the labels a, b, and c indicating data from primary human neutrophils, primary mouse neutrophils, and human pluripotent stem cell (hPSC)-derived neutrophils, respectively.

FIG. 2C shows a schematic diagram of the mechanism of L7Ae: kink-turn (k-turn) RNA-protein (RNP) switch (left), the structures of L7Ae eGFP microRNA223 (miR223)/microRNA142 (miR142) and k-turn tdTomato and CAR modRNA constructs (right).

FIG. 2D shows flow cytometry analysis of L7Ae-eGFP and tdTomato expression on SVG p12 brain cells and primary neutrophils treated with the indicated modRNA.

FIG. 2E shows schematic diagrams of various chlorotoxin (CLTX)-CAR modRNA vectors with human (hu) or mouse (ms) transmembrane ™, intracellular (intra), and/or CD3ζ signaling domains.

FIG. 2F shows cytotoxicity against U87MG glioblastoma cells at different ratios of neutrophil-to-tumor target using CAR modRNA-engineered primary human neutrophils at 24 hours. Control=A, exosome=E, CAR #1 (from FIG. 2E)=B, CAR #2 (from FIG. 2E)=D, and CAR #3 (from FIG. 2E)=C.

FIG. 2G shows cytotoxicity against U87MG glioblastoma cells at different ratios of neutrophil-to-tumor target using CAR modRNA-engineered primary mouse neutrophils at 24 hours. Control=A, exosome=E, CAR #1 (from FIG. 2E)=B, CAR #2 (from FIG. 2E)=D, and CAR #3 (from FIG. 2E)=C.

FIG. 2H shows cytotoxicity against U87 MG glioblastoma cells at a 10:1 ratio of neutrophil-to-tumor target using CAR modRNA-engineered primary human neutrophils loaded with R—SiO₂-tirapazamine (TPZ) or R—Si—O₂-temolozomide at 24 and 48 hours.

FIG. 2I shows the in vivo distribution of eGFP modRNA engineered neutrophils in the indicated tissues/organs 24 hours after systemic administration of exosome/modRNA complex into wild-type mice with the labels A, B, and C indicating data from phosphate-buffered saline (PBS), LNP-eGFP modRNA, and exosome-eGFP modRNA, respectively.

FIG. 2J shows a schematic diagram of systemically administered, tdTomato modRNA, and CLTX modRNA for a proof-of-concept in vivo tumor-killing study.

FIG. 2K shows quantification of the time-dependent tumor burden by bioluminescent imaging (BLI) at the indicated days for immunodeficient NOD.Cg-RAG^{1tm1Mom}IL2rg^{tm1Wjl}/Sz.J (NRG) mice, into the right forebrain of which were stereotypically implanted luciferase (Luci)-expressing U87MG cells (5×10⁵) and, after four days, were intravenously treated with the indicated modRNAs weekly for six weeks. Data are mean±s.d. (n=5).

FIG. 2L shows a Kaplan-Meier curve demonstrating survival of the indicated experimental groups.

FIG. 3A shows a schematic diagram of intravenously administered PBS (control group) or modRNA CARs (treatment group) for a study assessing the resultant neutrophil CARs' (Neu-CARs) anti-tumor activities and in vivo tumor-killing efficacy in a GL261 syngeneic mouse model.

FIG. 3B shows a representative flow cytometry analysis of Ly6G and CAR (equating with IgG4) expression in peripheral blood cells taken from the mice in the study described in FIG. 3A.

FIG. 3C is the BLI of tumor burden in syngeneic mice at the indicated days after treated with PBS, tdTomato, or CLTX CAR modRNAs (n=5), wherein the white circles indicate luminescence in the 10⁴ range, the white rectangles indicate luminescence with an exterior band in the 10⁴ range and the interior in the 10⁵ range, the white diamonds indicate luminescence with an exterior band in the 10⁴ range and the interior in the 10⁷-10⁸ range, and the white hexagons indicate luminescence with an exterior band in the 10⁴ range and the interior in the 10⁹ range.

FIG. 3D shows a quantification of the time-dependent tumor burden by BLI at the indicated days for syngeneic mice, into the right forebrain of which were stereotypically implanted luciferase (Luci)-expressing GL261 cells (5×10⁵) and, after four days, were intravenously treated with the indicated modRNAs weekly for four weeks. Data are mean±s.d. (n=5).

FIG. 3E shows a Kaplan-Meier curve demonstrating survival of the indicated groups (i.e., PBS, tdTomato modRNA or CAR modRNA) (n=5).

FIG. 4A shows a schematic diagram of intravenously administered PBS (control group), modRNA tdTomato, or CARs (the latter two, the treatment groups) for a study assessing the resultant neutrophil CARs' (Neu-CARs) anti-tumor activities and the in vivo tumor-killing efficacy in a humanized mouse model.

FIG. 4B shows quantification of human CD45⁺ cells in mouse peripheral blood in different experimental groups.

FIG. 4C shows quantification of the time-dependent tumor burden by BLI at the indicated days for humanized NRG mice, into the right forebrain of which were stereotypically implanted luciferase (Luci)-expressing U87MG cells (5×10⁵) and, after four days, were intravenously treated with the indicated modRNAs weekly for six weeks. Data are mean±s.d. (n=5).

FIG. 4D shows a Kaplan-Meier curve demonstrating survival of the indicated experimental groups.

FIG. 4E shows quantification of released human tumor necrosis factor-α (TNFα) and IL-6 in the peripheral blood of different mouse groups at the indicated days. Data are mean±SD, n=5.

FIG. 5A shows quantification of tumor lysis against GL261 cells by modRNA CAR neutrophils, chemo-drug Temozolomide (TMZ), or both.

FIG. 5B shows a schematic diagram of intraperitoneally (i.p.) administered PBS (control group) or β-Glucan (treatment groups) for a study assessing neutrophil loss after TMZ treatment in mice.

FIG. 5C shows quantification of neutrophils in mouse peripheral blood at the indicated days.

FIG. 5D shows a schematic diagram of intravenously administered PBS (control group), TMZ, or modRNA CARs with or without TMZ (treatment group).

FIG. 5E shows quantification of the time-dependent tumor burden by BLI at the indicated days for syngeneic mice. Data are mean±s.d. (n=5).

FIG. 5F shows a Kaplan-Meier curve demonstrating survival of the indicated experimental groups.

FIG. 6A shows a schematic diagram of intravenously administered PBS (control group) or modRNA CARs (treatment group) for a safety study assessing modRNA CAR delivery to circulating neutrophils in vivo in a healthy dog model.

FIG. 6B shows a representative flow cytometry analysis of Ly6G and CAR (equating with IgG4) expression in peripheral blood cells taken from the canines in the study described in FIG. 6A.

FIG. 6C is graphical data relating to the measurement of albumin globulin (AG), CO2, aspartate transferase (ALT), and body weight in the dogs in the treatment cohort both before and after modRNA CAR treatment (n=6).

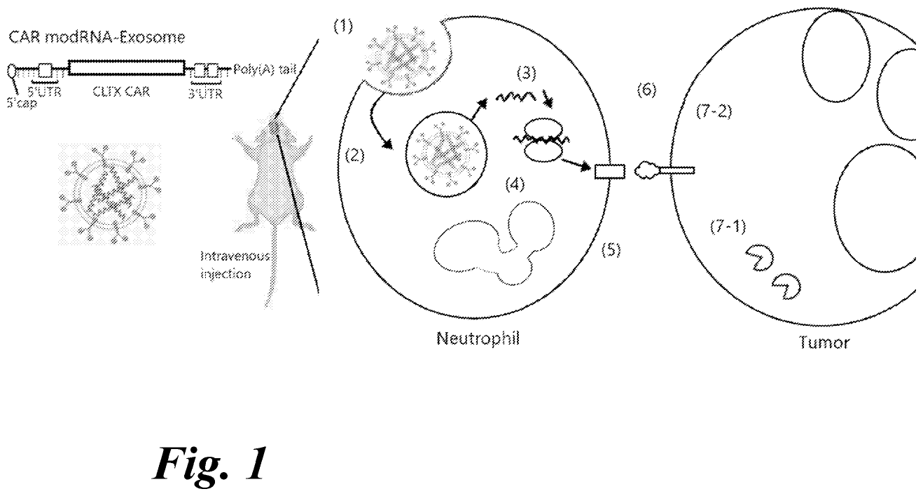

### DETAILED DESCRIPTION

While the concepts of the present disclosure are illustrated and described in detail in the description herein, results in the description are to be considered as exemplary and not restrictive in character; it being understood that only the illustrative embodiments are shown and described and that all changes and modifications that come within the spirit of the disclosure are desired to be protected.

Given their innate immunity against pathogens and native ability to cross physiological barriers, the present disclosure relates to neutrophils (e.g., human neutrophils) engineered with synthetic chimeric antigen receptors (CARs) and methods of producing and utilizing the same.

The present disclosure is predicated, at least in part, on the discovery of a method for producing CAR-neutrophils in vivo or ex vivo by systemically administering synthetic modified mRNA (modRNA) to an animal (i.e., in vivo), such as a mammal, or to isolated neutrophils (i.e., ex vivo/in vitro). modRNA is coined “modified” because chemically modified nucleotides are used during in vitro transcription; introduction of unmodified mRNA into mammalian cells can be unstable and trigger a cellular immune response, whereas modRNA is more stable and less immunogenic (see, e.g., Hadas et al., Molecular Therapy Methods Clinical Developments 14:300-305 (2019); Kariko et al., Immunity 23:165-175 (2005); and Kariko et al., Incorporation of psuedouridine into mRNA yields superior nonimmunogenic vector with increased translational capacity and biological stability, Molecular Therapies 16 (11): 1833-1840. (2008)). The method hereof is safe, potent and versatile. Superior anti-tumor activities have been displayed in mice and canine models. The method can be used to treat cancer, such as brain cancer, e.g., glioblastoma (GBM) and prostate cancer, and has the potential to treat other diseases.

modRNA Constructs and CAR-Expressing Neutrophils

In view of the above, modRNA constructs are provided, as are CAR-expressing neutrophils (or CAR neutrophils). In certain embodiments, the modRNA construts are neutrophil-specific. The term “CAR neutrophils” means neutrophils that have been modified through molecular biological methods to express a CAR on the surfaces of the neutrophils. Such engineered CAR-neutrophils can have striking anti-tumor activities and, in certain embodiments, can be used to treat and, optionally target, various disease states, including GBM.

A modRNA construct can comprise a nucleotide sequence encoding at least one archaeal ribosomal protein L7Ae and at least one neutrophil-specific microRNA recognition sequence (the “L7Ae modRNA construct”). The at least one neutrophil-specific microRNA recognition sequence in the L7Ae modRNA construct can be downstream of the nucleotide sequence encoding L7Ae.

L7Ae is a neutrophil-specific L7Ae: kink turn (k-turn) ribonucleoprotein (RNP) switch. A k-turn is a conserved RNA structural motif that typically comprises two stems (one with tandem A•G base pairs, the other with Watson-Crick pairs) linked by an asymmetric internal loop (or a sharp kink in the RNA helix). The internal loop can, for example, comprise a three-nucleotide bulge. In the absence of protein or metal ions, the k-turns can be unfolded in an extended conformation. However, upon binding an L7Ae-class protein, the RNA can adopt the kinked conformation. K-turns can play crucial roles in RNA folding, stability, and interactions with proteins.

L7Ae can recognize conventional k-turns in ribosomal and box C/D RNAs and bind specifically to some box H/ACA RNAs at terminal stem loops (these can have the A•G paired stem, but lack the Watson-Crick stem, for example).

As an RNP switch, an L7Ae: k-turn interaction can be used to construct translational regulators under control of an input protein that regulates the expression of desired output proteins. In other words, L7Ae can be used to strategically bind at a translation start site to block the progression of the ribosome during translation of mRNA into protein. Accordingly, the k-turn RNP comprises a synthetic regulatory circuit that can be used to provide a means to control cell behavior while avoiding potentially harmful genomic integration in therapeutic applications. This can be used to create post-transcriptional circuits via RNA-binding proteins, which can then be wired in a plug-and-play fashion to create networks of higher complexity.

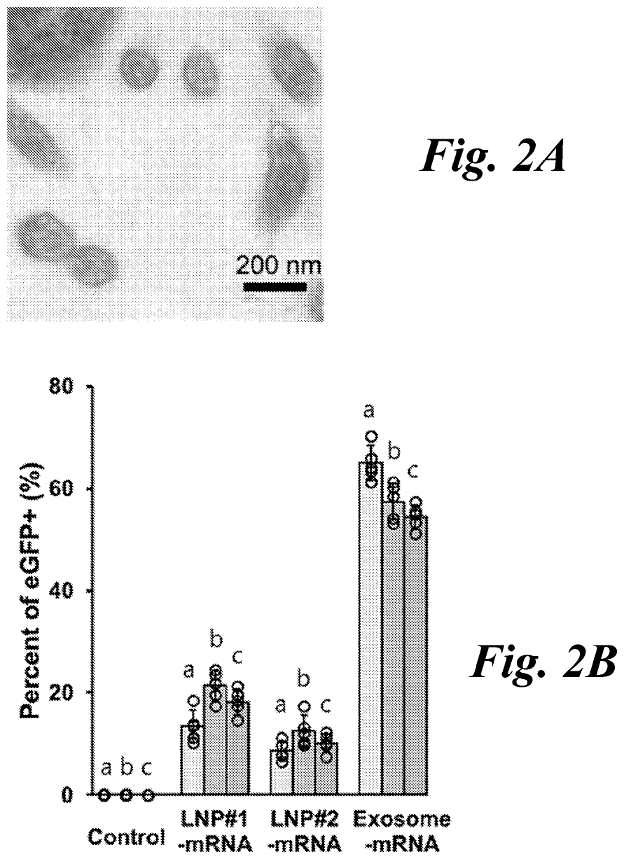

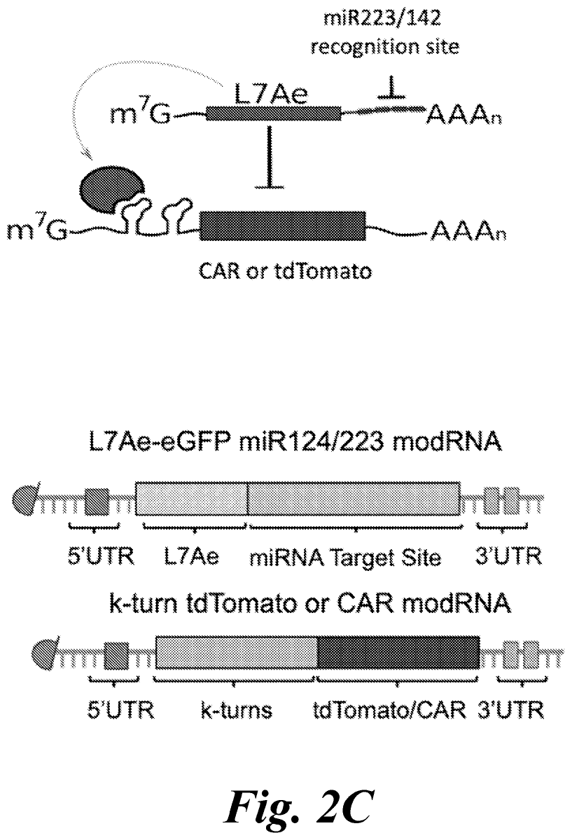

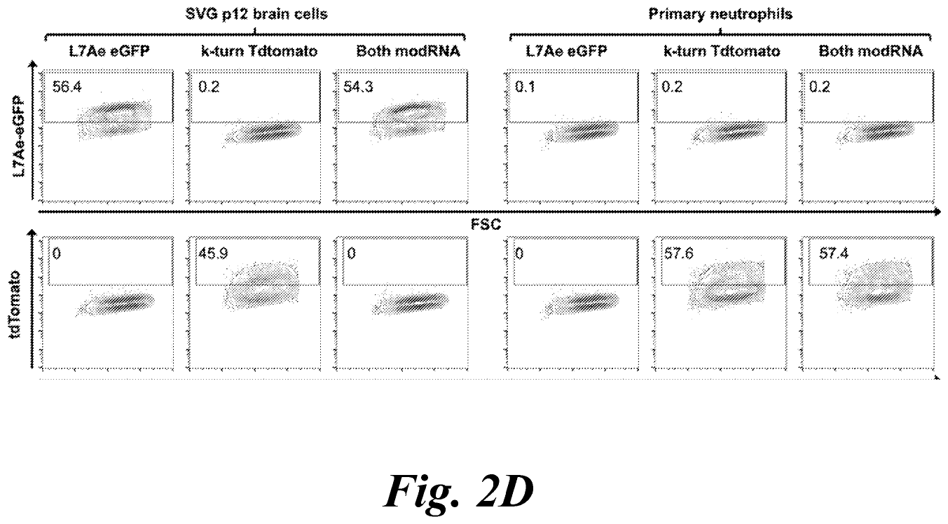

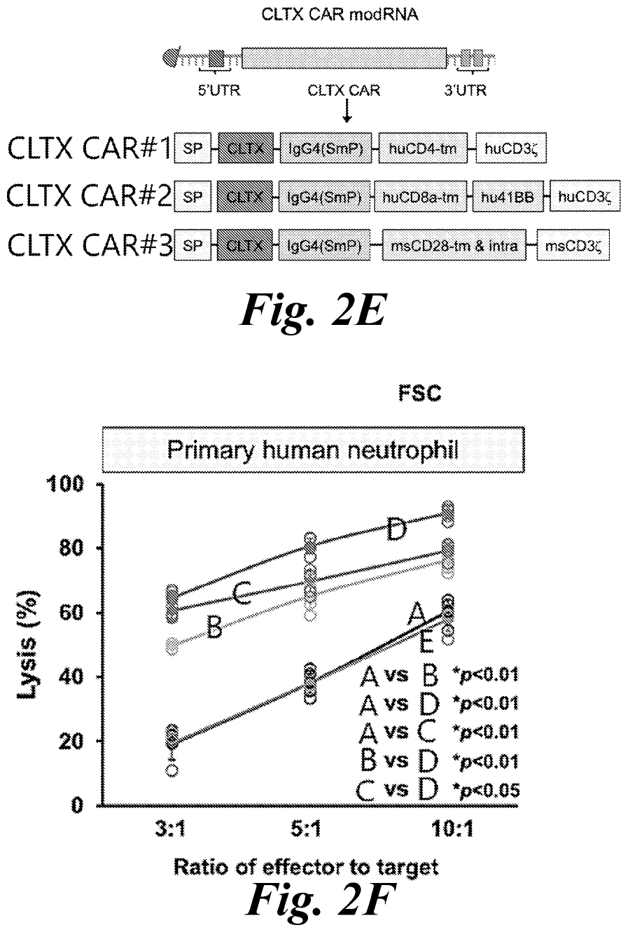

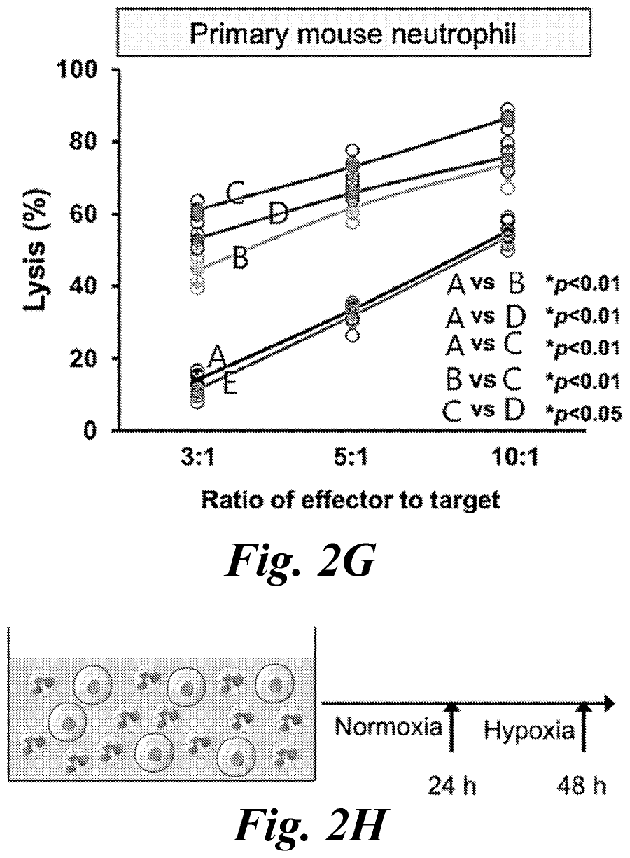

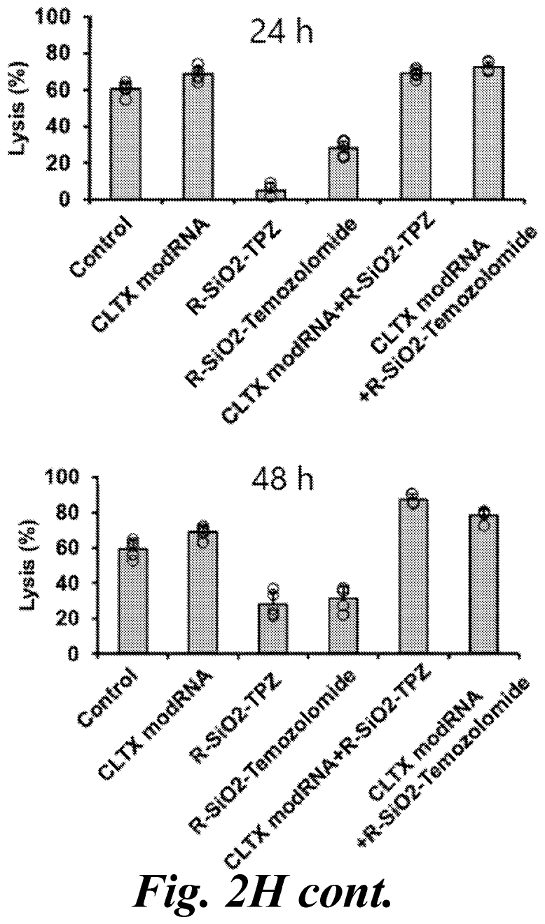

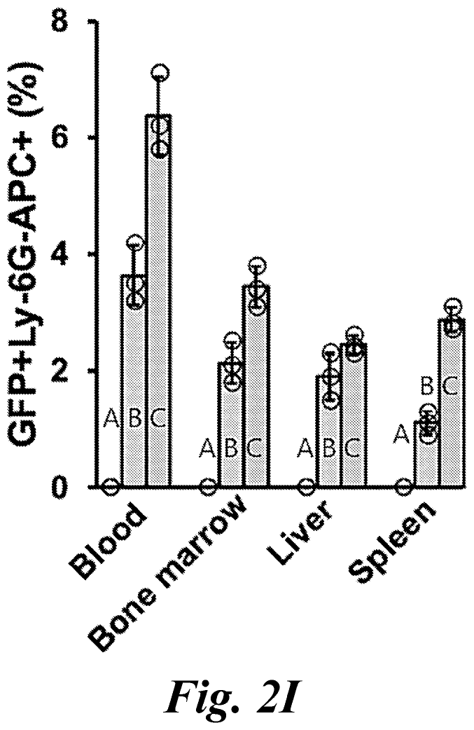

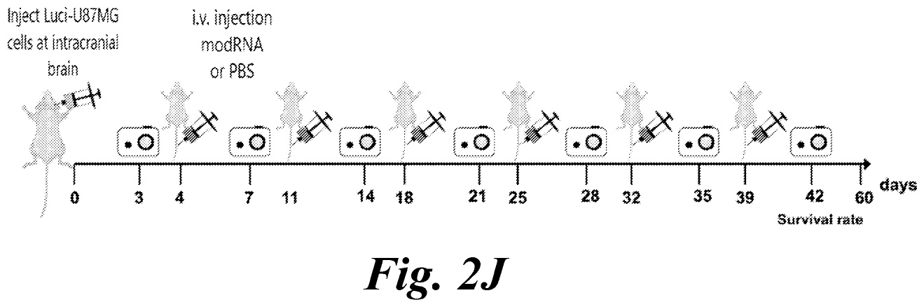

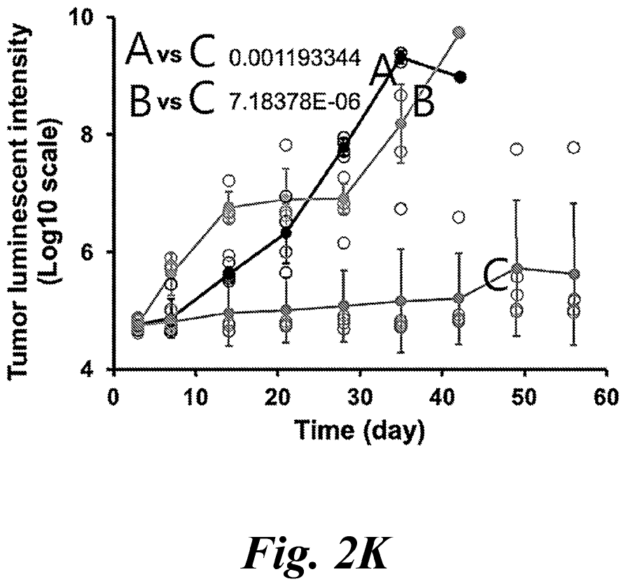

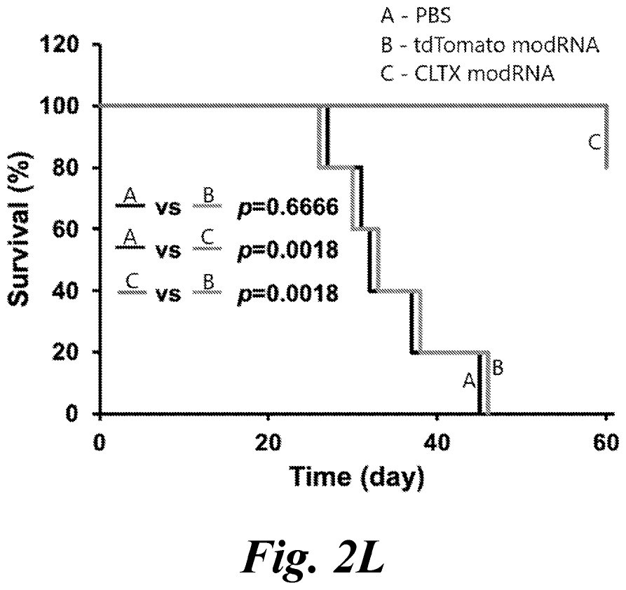

In certain embodiments, the L7Ae encoded by a modRNA construct hereof further comprises one or more neutrophil-specific microRNA recognition sequences. The neutrophil-specific microRNA recognition sequence can, for example, encode microRNA 223 (miR-223) or microRNA 142 (miR-142) binding sequences (see, e.g., FIG. 2C). Wroblewska et al., Mammalian synthetic circuits with RNA binding proteins for RNA-only delivery, Nature Biotechnology 33:839-841 (2015). In certain embodiments, the modRNA construct comprises a first neutrophil-specific microRNA recognition sequence encoding miR-223 and a second neutrophil-specific microRNA recognition sequence encoding miR-144. miR-223 and miR-142 are specifically expressed (or most enriched) in neutrophils, but not in other cells. This binding cassette (i.e., the modRNA construct hereof) allows miR-223 and miR-142 to bind and destroy L7Ae modRNA in nuetorphils, which can lead to expression of the other modRNA of interest in neutorphils (e.g., CAR, tdTomato in FIG. 2D). In non-neutrophil cells, L7A3 can be expressed (due to the lack of miR-223/miR-142), and the L7Ae can destroy the other modRNA of interest (e.g., CAR, tdTomato) and, thus, these target proteins will not express in non-neutrophil cells.

In certain embodiments, the neutrophil-specific mIR recognition sequence that encodes a binding sequence can be or comprise SEQ ID NO: 3, SEQ. ID NO: 4, SEQ ID NO: 5, SEQ ID NO: 6, SEQ ID NO: 7, or a functional variant of SEQ ID NO: 3, SEQ. ID NO: 4, SEQ ID NO: 5, SEQ ID NO: 6, or SEQ ID NO: 7. The term “functional variant” refers to a nucleotide, peptide, a polypeptide, or a protein having substantial or significant sequence identity or similarity to the reference nucleotide, peptide or polypeptide, which functional variant retains the biological activity of the reference sequence of which it is a variant. Functional variants encompass, for example, those variants of a sequence (the parent sequence) that retain the ability to exhibit the properties (such as, for example, binding functionality) and to a similar extent, the same extent, or to a higher extent, as the parent sequence. In reference to a nucleic acid sequence encoding the peptide or polypeptide, in some embodiments a nucleic acid sequence encoding a functional variant of the peptide or is about 10% identical, about 25% identical, about 30% identical, about 50% identical, about 65% identical, about 75% identical, about 80% identical, about 90% identical, about 95% identical, or about 99% identical to the nucleic acid sequence encoding the parent sequence.

A modRNA construct hereof can comprise (a) a nucleotide sequence encoding a CAR, and (b) at least one k-turn (the “modRNA CAR construct”). As noted above, L7Ae can recognize and bind to k-turn sequences.

In certain embodiments, the sequence encoding the at least one k-turn of the modRNA construct is upstream of the nucleotide sequence encoding the CAR. The modRNA CAR construct can comprise a nucleotide sequence encoding two k-turns. The two k-turns can be related by two-fold symmetry (the 2K unit) and can assemble into a variety of crystal lattices as two, three or four 2K units. The modRNA CAR construct can comprise a nucleotide sequence encoding three k-turns. The modRNA CAR construct can comprise a nucleotide sequence encoding four or more k-turns.

In certain embodiments, the sequence encoding the at least one k-turn of the modRNA construct is or comprises SEQ ID NO: 1, SEQ ID NO: 2, or a functional variant of SEQ ID NO: 1 or SEQ ID NO: 2. In certain embodiments, the sequence encoding the at least one k-turn of the modRNA construct is or comprises SEQ ID NO: 9 or a functional variant of SEQ ID NO: 9.

In certain embodiments, the sequence encoding the CAR comprises SEQ ID NO: 10, SEQ ID NO: 11, SEQ ID NO: 12, or a functional variant of SEQ ID NO: 10, SEQ ID NO: 11, or SEQ ID NO: 12.

The CAR encoded by the modRNA CAR construct can be any suitable CAR as known in the art. CARs are artificially constructed hybrid receptor proteins or polypeptides that can graft an arbitrary specificity onto an immune effector cell, such as a natural killer (NK) cell. See, e.g., Sadelain et al., “The Basic Principles of Chimeric Antigen Receptor Design,” Cancer Discovery OF1-11 (2013).

A CAR can comprise complementarity-determining regions (CDRs) to define its specificity. Accordingly, the portion of the modRNA CAR construct encoding the CAR can comprise a sequence encoding one or more CDRs.

Non-limiting examples of CDRs include, but are not limited to, CD19 (U.S. Pat. No. 7,446,190 and U.S. Patent Application Publication No. 2013/0071414), HER2 (Ahmed et al., Her2-specific T cells target primary glioblastoma stem cells and induce regression of autologous experimental tumors, Clinical Cancer Research 16 (2): 474-485 (2010)), MUC16 (Chekmasova et al., Successful eradication of established peritoneal ovarian tumors in SCID-Beige mice following adoptive transfer of T cells genetically targeted to the MUC16 antigen, Clinical Cancer Research 16 (14): 3594-3606 (2011)), and prostate-specific membrane antigen (PSMA) (Zhong et al., Chimeric antigen receptors combining 4-1BB and CD28 signaling domains augment PI3kinase/AKT/Bcl-XL activation and CD8+ T cell-mediated tumor eradication, Molecular Therapies 18 (2): 413-410 (2010)).

The CAR can have a pre-defined binding specificity to a desired target, such as matrix metallopeptidase 2 (MMP2), e.g., MMP2 on a glioma, such as a GBM. This can reduce or obviate off-target effects. The terms “polypeptide,” “peptide,” and “protein” are used interchangeably herein (unless expressly stated otherwise) to refer to a polymer of amino acid residues, a polypeptide, or a fragment of a polypeptide, peptide, or fusion polypeptide. The terms apply to amino acid polymers in which one or more amino acid residue is an artificial chemical mimetic of a corresponding naturally occurring amino acid, as well as to naturally occurring amino acid polymers and non-naturally occurring amino acid polymers.

Generally, a CAR is a fusion protein that can comprise a recognition region, co-stimulation domains, various signaling domains, costimulatory domains, spacers, and/or hinges. For example, where desired, the CARs can also comprise additional elements, such as a signal peptide to ensure proper export of the fusion protein to the cell surface, a transmembrane domain to ensure the fusion protein is maintained as an integral membrane protein, and a hinge domain for imparting flexibility to the recognition region and facilitating strong binding to the targeting moiety. Accordingly, the portion of the modRNA CAR construct encoding the CAR can comprise a sequence encoding a recognition region, co-stimulation domains, various signaling domains, costimulatory domains, spacers, and/or hinges. Desirably, the CAR is suitable for using the CAR neutrophils to treat cancer, e.g., the CAR binds a cell-surface antigen on a cancerous cell with high specificity.

The use of terms and phrases with regard to CAR binding specificity, such as “binds with specificity,” “binds with high affinity,” “binds with high specificity,” or “specifically” or “selectively” binds, indicates a binding reaction between a CAR, such as a CAR comprising CLTX, on a neutrophil and a target molecule, such as a protein (e.g., a receptor, an enzyme (e.g., MMP2), or a cell-surface marker) that is present on a targeted cell, such as a cancerous cell (e.g., a cell of which a tumor is comprised) or other diseased cell. Thus, under binding conditions that are conducive to facilitate or otherwise promote binding of a CAR neutrophil with a target molecule that is present on a targeted cell, such as a cancerous cell or other diseased cell, such a CAR neutrophil does not bind significantly, if at all, to other molecules, such as proteins (e.g., receptors, enzymes, and cell-surface markers) present on normal, healthy cells. Specific binding or binding with high affinity can be at least 25% greater, more often at least 50% greater, most often at least 100% (2-fold) greater, normally at least ten times greater, more normally at least 20-times greater, and most normally at least 100-times greater than the binding of any other non-targeted molecule.

In certain embodiments, the CARs hereof bind with high specificity to a cancer cell (e.g., a brain cancer cell). In certain embodiments, the CARs hereof bind with high specificity to beta amyloid (e.g., for use in targeting/treating a neurological disorder). The CAR can be designed, for example, to target beta amyloid (e.g., soluble oligomers of the amyloid-β peptide (AβOs)). As the accumulation of AβOs in the brain has been implicated in synapse failure and memory impairment in Alzheimer's disease, targeting such AβOs can be useful in effectively delivering nanoparticle/drug cargo thereto to, for example, treat Alzheimer's disease and/or symptoms associated therewith. Selles et al., AAV-mediated neuronal expression of an scFv antibody selective for Aβ oligomers protects synapses and rescues memory in Alzheimer models, Molecular Therapies 31 (2): 409-419 (2023). In certain embodiments, the CAR can comprise a NUscl single chain variable fragment (scFv) that selectively targets a population of AβOs in a subject. In certain embodiments, the CAR comprises a scFv.

The modRNA CAR construct can encode CARs that include an extracellular domain, a transmembrane domain, and/or an intracellular domain. The extracellular domain can include an antigen binding/recognition region/domain. The antigen binding domain of the CAR can bind to a specific antigen, such as a cancer/tumor antigen (e.g., for the treatment of cancer), a pathogenic antigen, such as a viral antigen (e.g., for the treatment of a viral infection), or a CD antigen. In certain embodiments, the CAR comprises a ligand that targets a cancer. The cancer can be brain cancer. The ligand can be a tumor-targeting ligand.

Examples of tumor antigens include, but are not limited to, carbonic anhydrase IX (CAIX), carcinoembryonic antigen (CEA), CD5, CD7, CD10, CD19, CD20, CD22, CD30, CD33, CD34, CD38, CD41, CD44, CD49f, CD56, CD74, CD123, CD133, CD138, an antigen of a cytomegalovirus infected cell (e.g., a cell surface antigen), epithelial glycoprotein 2 (EGP2), epithelial glycoprotein 40 (EGP40), epithelial cell adhesion molecule (EpCAM), receptor tyrosine-protein kinase erb-B2, 3 or 4, folate-binding protein (FBP), fetal acetylcholine receptor (AChR), folate receptor α (FRα), folate receptor β (FRβ), ganglioside G2 (GD2), ganglioside G3 (GD3), human epidermal growth factor receptor 2 (HER2), human telomerase reverse transcriptase (hTERT), interleukin 13 (IL-13) receptor subunit α2 (IL-13Rα2), K light chain, kinase insert domain receptor (IDR), Lewis A (CA19.9), Lewis Y (LeY), L1 cell adhesion molecule (L1CAM), melanoma antigen family A1 (MAGE-A1), mucin 16 (Muc-16), mucin 1 (Muc-1), mesothelin (MSLN), NKG2D ligand, cancer-testis antigen NY-ESO-1, oncofetal antigen (h5T4), prostate stem cell antigen (PSCA), prostate-specific membrane antigen (PSMA), tumor-associated glycoprotein 72 (TAG-72), vascular endothelial growth factor receptor (VEGF-R, such as R2), and Wilms tumor protein (Wt-1).

The cancer can be glioblastoma. Where the cancer is glioblastoma, the ligand (i.e., the ligand encoded by the modRNA CAR construct) can, for example, be or comprise a CAR comprising a 36-amino acid GBM-targeting chlorotoxin (CLTX) peptide or other brain tumor-targeting ligand (e.g., IL-13) or a scFv.

The cancer can be prostate cancer. In certain embodiments, the ligand that targets a cancer is PSMA. The CAR can comprise a ligand that targets PSMA. The PSMA-targeting ligand can be a derivative of pentanedioic acid (see, e.g., U.S. Pat. Nos. 5,968,915, 5,863,536, 5,795,877, 5,902,817, and Majer et al., Synthesis and biological evaluation of thiol-based inhibitors of glutamate carboxypeptidase II: discovery of an orally active GCP II inhibitor, J Medicinal Chemistry 46 (10): 1989-1996 (2003), all of which are hereby specifically incorporated by reference for their teachings regarding same). Other PSMA-targeting ligands include MUPA, DUPA, and peptide analogs, such as quisqualic acid, Asp-Glu, Glu-Glu, Gly-Glu, γ-Glu-Glu, β-N-acetyl-L-Asp-L-Glu, and the like. In certain embodiments, the modRNA CAR construct encodes a CAR comprising a ligand that targets a PSMA-specific membrane antigen.

The CAR can comprise a transmembrane domain. In certain embodiments, the CAR comprises a neutrophil-specific transmembrane domain. Examples of transmembrane domains include, but are not limited to, a CD3-zeta (CD3ζ) polypeptide, a CD4 polypeptide, a CD8 polypeptide, a CD28 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, and a BTLA polypeptide. In certain embodiments, the modRNA CAR construct encodes a CAR comprising a CD8 transmembrane domain. In certain embodiments, the modRNA CAR construct encodes a CAR comprising a human CD8 transmembrane domain. The neutrophil-specific transmembrane domain can be CD32a. The neutrophil-specific transmembrane domain can be CD4. The neutrophil-specific transmembrane domain can be NKG2D, Dectin-1, an IL-6 receptor, or CD16.

The CAR can comprise an intracellular domain. The intracellular domain can comprise, for example, a CD3 (polypeptide, and can further comprise at least one costimulatory signaling region comprising at least one costimulatory molecule or domain. “Costimulatory molecule” or “costimulatory domain” refers to a cell surface molecule, other than an antigen receptor/ligand required for an efficient response of lymphocytes to antigen. The CAR can comprise a neutrophil-specific costimulatory domain. In certain embodiments, the costimulatory domain can comprise a CD28 polypeptide, a 4-1 BB polypeptide, an OX40 polypeptide, an ICOS polypeptide, a DAP-10 polypeptide, a PD-1 polypeptide, a LAG-3 polypeptide, a 2B4 polypeptide, a BTLA polypeptide, or a CTLA-4 polypeptide. In certain embodiments, the modRNA CAR construct encodes a CAR comprising a 41BB costimulatory domain. In certain embodiments, the modRNA CAR construct encodes a CAR comprising a human 41BB costimulatory domain. In certain embodiments, the CAR comprises a neutrophil-specific co-stimulatory domain that is or comprises a 41BB co-stimulatory domain or a CD28 polypeptide. In certain embodiments, the modRNA CAR construct encodes a CAR comprising a CD3ζ intracellular domain. In certain embodiments, the modRNA CAR construct encodes a CAR comprising a human CD3ζ intracellular domain.

modRNA constructs encoding the CARs can be prepared using genetic engineering techniques as known in the art (see, e.g., Sambrook et al., Molecular Cloning: A Laboratory Manual, 3^{rd} ed., Cold Spring Harbor Laboratory Press (2001) and Green & Sambrook, Molecular Cloning: A Laboratory Manual, 4^{th} ed., Cold Spring Harbor Laboratory Press (2012), both of which are specifically incorporated herein by reference for their teachings regarding same) and exemplified herein. In certain embodiments, the placement of the recognition region in the fusion protein is generally such that display of the region on the exterior of the neutrophil is achieved.

The CAR can further be engineered to comprise a neutrophil-specific transmembrane domain. The neutrophil-specific transmembrane domain can be a TLR4 polypeptide, a TLR2 polypeptide, a MET polypeptide, a granulocyte colony stimulating factor receptor (G-CSFR), a Myd88 polypeptide, a TRIF polypeptide, a Syk peptide, a CD40 polypeptide, CD32a, Dectin-1, a IL-6 receptor (IL6R), an Fc Epsilon Receptor Ig (FCER1G) polypeptide, a toll-like receptor 7 (TLR7), a CD16 transmembrane polypeptide, a CD8 polypeptide, a CD28 polypeptide, an OX40 polypeptide, an ICOS polypeptide, a CTLA-4 polypeptide, a PD-1 polypeptide, a LAG-3 polypeptide, a BTLA polypeptide, a natural killer group 2D (NKG2D), Dectin-1, CD16 or other Fcγ receptors.

The transmembrane (tm), intracellular co-stimulatory (intra) and/or signaling domains of a CAR can further be optimized, for example, as desired. In certain embodiments, the transmembrane, intracellular co-stimulatory, and signaling domains of a CAR are optimized using human CD4-tm, CD8a-tm, 41BB, and CD3ζ or mouse CD28-tm, CD28-intra, and CD3ζ, respectively (see, e.g., FIG. 2E). In certain embodiments, when the subject is a human, the CAR comprises a human CD4 transmembrane domain and a human CD3ζ intracellular domain. In certain embodiments, the CAR comprises a human CD8 transmembrane domain, a human 41BB co-stimulatory domain, and a human CD3ζ intracellular domain.

The CAR can comprise a GBM-targeting peptide, such as a 36-amino acid GBM-targeting CLTX peptide. In such embodiments, the GBM-targeting peptide (e.g., the 36-amino acid GBM-targeting CLTX peptide) can be coupled with a CD4 transmembrane domain and a CD3ζ intracellular domain, such that the CAR comprises a 36-amino acid GBM-targeting chlorotoxin peptide, a CD4 transmembrane domain, and a CD3ζ intracellular domain. Accordingly, in certain embodiments, the modRNA CAR construct encodes a CAR comprising a 36-amino acid GBM-targeting CLTX peptide, a CD4 transmembrane domain, and a CD3ζ intracellular domain.

In other embodiments, the GBM-targeting peptide (e.g., the 36-amino acid GBM-targeting chlorotoxin peptide) can be coupled with (i) either of a CD32a transmembrane domain or a CD16 transmembrane domain, and (ii) a CD3ζ intracellular signaling domain, such that the CAR comprises (i) a 36-amino acid GBM-targeting chlorotoxin peptide, (ii) a CD32a transmembrane domain or a CD16 transmembrane domain, and (iii) a CD3ζ intracellular signaling domain. Accordingly, the modRNA CAR construct can encode a CAR comprising (i) a 36-amino acid GBM-targeting CLTX peptide, (ii) either a CD32a transmembrane domain or a CD16 transmembrane domain, and (iii) a CD3ζ intracellular domain.

In yet other embodiments, the GBM-targeting peptide (e.g., the 36-amino acid GBM-targeting chlorotoxin peptide) can be coupled with (i) either of a CD32a transmembrane domain or a CD16 transmembrane domain and (ii) a CD3ζ intracellular signaling domain, such that the CAR comprises (i) a 36-amino acid GBM-targeting chlorotoxin peptide, (ii) either of a CD32a transmembrane domain or a CD16 transmembrane domain, and (iii) a CD3ζ intracellular signaling domain. Accordingly, in certain embodiments, the modRNA CAR construct encodes a CAR comprising (i) a 36-amino acid GBM-targeting CLTX peptide, (ii) either of a CD32a transmembrane domain or a CD16 transmembrane domain, and (iii) a CD3ζ intracellular domain.

In yet still other embodiments, the GBM-targeting peptide (e.g., the 36-amino acid GBM-targeting chlorotoxin peptide) can be coupled with (i) either of a CD32a transmembrane domain or a CD16 transmembrane domain and (ii) either of a CD32aγ intracellular signaling domain or a CD16 intracellular signaling domain, alone or in further combination with (iii) a CD3ζ intracellular signaling domain, such that the CAR comprises (i) a 36-amino acid GBM-targeting chlorotoxin peptide, (ii) either of a CD32a transmembrane domain or a CD16 transmembrane domain and (iii) either of a CD32aγ intracellular signaling domain or a CD16 intracellular signaling domain, alone or in further combination with (iv) a CD3ζ intracellular signaling domain. Accordingly, in certain embodiments, the modRNA CAR construct encodes a CAR comprising (i) a 36-amino acid GBM-targeting CLTX peptide, and (ii) either of a CD32a transmembrane domain or a CD16 transmembrane domain, alone or in further combination with (iii) a CD3ζ intracellular signaling domain.

In yet even still other embodiments, the GBM-targeting peptide (e.g., the 36-amino acid GBM-targeting chlorotoxin peptide) can be coupled with a NKG2D transmembrane domain, a 2B4 co-stimulatory domain, and a CD3ζ intracellular signaling domain, such that the CAR comprises a 36-amino acid GBM-targeting chlorotoxin peptide, a NKG2D transmembrane domain, a 2B4 co-stimulatory domain, and a CD3ζ intracellular signaling domain. Accordingly, in certain embodiments, the modRNA CAR construct encodes a CAR comprising (i) a 36-amino acid GBM-targeting CLTX peptide, (ii) a NKG2D transmembrane domain, (iii) a 2B4 co-stimulatory domain, and (iv) a CD3ζ intracellular signaling domain.

In various other embodiments, such as when the CAR-expressing neutrophils are for use in treatment cancer (e.g., for use in the preparation of a medicament for treating cancer), the CAR can comprise an IL-13 receptor α2 (IL-13Rα2)-targeted quadruple mutant IL-13 (TQM13) T-CAR, GD-2 targeting scFV, HER2-targeting scFV, EGFRvIII-targeting scFV, or other glioma-targeting scFVs, CD4 transmembrane domain, and a CD3ζ intracellular signaling domain. Accordingly, in certain embodiments, the modRNA CAR construct (e.g., where the resulting CAR-expressing neutrophils are for use in treating cancer or preparation of a medicament for treating cancer) encodes a TQM13 T-CAR. In certain embodiments, the modRNA CAR construct (e.g., where the resulting CAR-expressing neutrophils are for use in treating cancer or preparation of a medicament for treating cancer) encodes a CAR comprising (i) a TQM13-targeting peptide or scFv, a GD-2 targeting scFv, a HER2-targeting scFv, EGFRvIII-targeting scFv, or other glioma-targeting scFvs, (ii) a CD4 transmembrane domain, and (iii) a CD3ζ intracellular signaling domain.

The resulting coding region can be inserted into an expression vector for subsequent introduction into a recipient cell, such as a neutrophil. The term “vector” means any nucleic acid that functions to carry, harbor, or express a nucleic acid of interest. Nucleic acid vectors can have specialized functions, such as expression, packaging, pseudotyping, or transduction. Vectors can also have manipulatory functions if adapted for use as a cloning or shuttle vector. The structure of the vector can include any desired form that is feasible to make and desirable for a particular use. Such forms can include, for example, circular forms such as plasmids and phagemids, as well as linear or branched forms. A nucleic acid vector can be composed of, for example, DNA or RNA, as well as contain (partially or fully) nucleotide derivatives, analogs or mimetics. Such vectors can be obtained from natural sources, produced recombinantly or chemically synthesized.

By way of non-limiting examples, a plasmid or viral expression vector (e.g., a lentiviral vector, a retrovirus vector, sleeping beauty, and piggyback (transposon/transposase systems that include a non-viral mediated CAR gene delivery system)) can be prepared that encodes a fusion protein (i.e., modRNA CAR construct) comprising a recognition region, one or more co-stimulation domains, and an activation signaling domain, in frame and linked in a 5′ to 3′ direction.

In certain embodiments, the subject is a human and the CAR comprises SEQ ID NO: 10 or a functional variant thereof. In certain embodiments, the CAR comprises SEQ ID NO: 10 or a functional variant thereof. In certain embodiments, the subject is a mouse and the CAR comprises SEQ ID NO: 11 or a functional variant thereof. In certain embodiments, the CAR comprises SEQ ID NO: 11 or a functional variant thereof. In certain embodiments, the subject is a canine and the CAR comprises SEQ ID NO: 12 or a functional variant thereof. In certain embodiments, the CAR comprises SEQ ID NO: 12 or a functional variant thereof.

CAR expression can be driven using any suitable promoter, such as exemplified herein. Examples of promoters include, but are not limited to, various constitutive and inducible promoters, such as a constitutive CAG promoter, an EF1a promoter, a UBC constitutive promoter, or a Teton-3G inducible promoter.

The placement of the recognition region in the fusion protein/construct will generally be such that display of the region on the exterior of the neutrophil is achieved. Where desired, the CARs can also include additional elements, such as a signal peptide (e.g., CD8α signal peptide) to ensure proper export of the fusion protein to the cell surface, a transmembrane domain to ensure the fusion protein is maintained as an integral membrane protein (e.g., CD3ζ transmembrane domain), and a hinge domain that imparts flexibility to the recognition region and allows strong binding to the targeting moiety.

The modRNA constructs hereof can be used to produce stable CAR-expressing neutrophils that express the modRNA construct(s). Such neutrophils can exhibit anti-cancer (e.g., anti-GLB) activity in a hypoxic tumor microenvironment (TME), for example.

In certain embodiments, the modRNA constructs are encapsulated within an exosome, a microvesicle) or a lipid nanoparticle (LNP). As used herein, the term “encapsulate” and grammatical variants thereof refer to the process of coating one or more substances with another material (e.g., with sizes on a nano-scale). The encapsulated material is the “internal phase” or the “core.” The encapsulation material is the “external phase,” or the shell, coating, or membrane. In the present context, the external phase can comprise the exosome, microvesicle, or LNP outer membrane, which confines the internal phase (i.e., the payload (e.g., the modRNA constructs hereof)) and, optionally, other substances such as proteins, small molecule drugs (e.g., chemotherapy drugs), lipids, imaging agents, and/or aqueous/lipid emulsions.

Exosomes, microvesicles, and/or LNPs can be used to transport RNA, protein, and other substances therein for delivery of the modRNA constructs hereof (see, e.g., FIGS. 2F-2H). The term “exosome” means a cell-derived phospholipid membrane bound vesicle that has, for example, a diameter at or between about 30-500 nm (e.g., 30 nm to about 500 nm, about 30 nm to about 50 nm, about 30 nm to 500 nm, or 30-50 nm). Exosomes are typically present in biological fluids, including blood, urine, and cultured medium of cell cultures. Exosomes are either released from the cell when multivesicular bodies fuse with the plasma membrane or they are released directly from the plasma membrane. The term “exosome” also means and encompasses synthetic exosomes. “Synthetic exosome” means an exosome that is not naturally occurring.

The term “lipid nanoparticle” or “LNP” as used herein means a nano-scale particles comprising one or more lipids and/or phospholipids that can be used to encapsulate, or otherwise as a carrier for, a payload (e.g., the modRNA constructs hereof). Certain non-limiting examples of LNPs include rough silica nanoparticles, cytosine arabinoside-based liposomes (e.g., DepoCyt®), polyamidoamine (PAMAM) dendrimer-albumin nanoparticles, and fullerene (e.g., gadofullerenol/fullerenol). The rough silica nanoparticles can be biodegradable mesoporous organic silica.

As used herein, the term “microvesicle” generally means any plasma membrane bound particle that may reside within a cell, or in the extracellular environment. These structures are not limited in any way with regard to in vivo localization (e.g., intracellular or extracellular), in a body fluid, in a cell culture media, generated by in vitro cultured cells, mechanism of origin, or size characteristics. In some embodiments, a microvesicle can range in size with a lower size limit of at least about 0.1 μm in diameter, or alternatively, about 0.2 μm in diameter, about 0.3 μm in diameter, about 0.4 μm in diameter, about 0.5 μm in diameter, about 0.6 μm in diameter, about 0.7 μm in diameter, about 0.8 μm in diameter, about 0.9 μm in diameter, or about 1.0 μm in diameter. In some embodiments, a microvesicle has an upper size limit of not more than about 1.0 μm or micron, or alternatively, not more than about 1.5 μm, about 2.0 μm, or about 2.5 μm.

Each of exosomes, microvesicles, and LNPs can target and/or carry the payload encapsulated therein across cell membranes and biological barriers (such as, for example, the blood brain barrier (BBB)) to deliver the payload to a targeted site in a subject (e.g., the cytoplasm of a neutrophil). Exosomes, microvesicles, and LNPs can be, for example, administered systemically to a subject for delivery of the modRNA constructs hereof. As used herein, “delivery” means the administration and localization of an exosome, microvesicle, or LNP carrying a payload to target tissues or target cells of a subject.

The modRNA constructs hereof can be delivered via an exosome, microvesicle, or LNP to the cytoplasm of a target cell (e.g., a neutrophil). In certain embodiments, the modRNA constructs hereof can be delivered via an exosome, a microvesicle, or an LNP to a membrane of a target cell (e.g., a neutrophil). In some embodiments, the membrane of the exosome, microvesicle, or LNP fuses with a membrane of the target cell (e.g., a neutrophil).

In some embodiments, the exosome comprises a membrane that forms a particle that has a diameter of 30-100 nm, 30-200 nm, or 30-500 nm. In some embodiments, the exosome comprises a membrane that forms a particle that has a diameter of 10-100 nm, 20-100 nm, 30-100 nm, 40-100 nm, 50-100 nm, 60-100 nm, 70-100 nm, 80-100 nm, 90-100 nm, 100-200 nm, 100-150 nm, 150-200 nm, 100-250 nm, 250-500 nm, or 10-1000 nm. The ranges set forth in this paragraph are inclusive of the stated end points and all 1 nm increments contained therein.

In some embodiments, the membrane of a exosome or microvesicle comprises lipids and fatty acids. In some embodiments, the membrane comprises one or more of phospholipids, glycolipids, fatty acids, sphingolipids, phosphoglycerides, sterols, cholesterols, and phosphatidylserine. In addition, the membrane can comprise one or more polypeptides and one or more polysaccharides, such as glycans.

In some embodiments, the exosome and/or microvesicle is generated by a producer cell (or parental cell), such as, e.g., a mammalian cell. In some embodiments, the membrane of the exosome or microvesicle comprises one or more molecules derived from the producer cell. The exosome can be generated in a cell culture system and isolated (e.g., by separating the exosome from the producer cell) using processes well known in the art. Separation can be achieved by sedimentation. For example, an exosome can have a specific density between 0.5-2.0, 0.6-1.0, 0.7-1.0, 0.8-1.0, 0.9-1.0, 1.0-1.1, 1.1-1.2, 1.2-1.3, 1.4-1.5, 1.0-1.5, 1.5-2.0, and 1.0-2.0 kg/m³.

In certain embodiments, the exosome or microvesicle is synthetic. In some embodiments, the exosome or microvesicle is modified (e.g., by introducing a payload or otherwise modifying the content of the complex, such as by changing the protein, lipid or glycan content of the membrane). For example, exosomes are first isolated from a producer cell and then modified as desired, thereby generating synthetic exosomes. In some embodiments, the producer cell is modified. For example, an exogenous nucleic acid, an exogenous polypeptide or small molecule or toxin can be introduced into the producer cell. Alternatively or in addition, the producer cell can otherwise be modified (e.g., by modifying the cellular or membrane content, such as by changing the lipid or glycan content of the cell membrane). Exosomes and microvesicles generated from the modified producer cells comprise one or more of the modifications of the producer cell. The process produces synthetic exosomes or microvesicles. In some embodiments, both the producer cell and the exosome or microvesicle isolated from the producer cell are modified as described herein. The term “isolated” means that the material is removed from its original environment, e.g., the natural environment if it is naturally occurring. For example, a naturally occurring neutrophil present within a living organism is not isolated, but the same neutrophil separated from some or all the coexisting materials in the natural system is isolated.

In some embodiments, the exosome, microvesicle, or LNP delivers the payload (i.e., modRNA constructs) to a target (e.g., a neutrophil). The payload can comprise the modRNA constructs, which can be internalized into the target (e.g., a neutrophil) that is contacted with the exosome, microvesicle, or LNP. Contacting can occur in vitro or in a subject (e.g., in vivo). The modRNA constructs can be introduced into an exosome, microvesicle, or LNP.

The exosome, microvesicle, or LNP can interact with the target cell via membrane fusion and deliver the modRNA constructs loaded within an exosome, microvesicle, or LNP composition to the surface or cytoplasm of a target cell. In some embodiments, membrane fusion occurs between the exosome/microvesicle/LNP and the plasma membrane of a target cell. In other embodiments, membrane fusion occurs between the exosome/microvesicle/LNP and an endosomal membrane of a target cell.

In some embodiments, the exosome, microvesicle, or LNP comprises polypeptides on its surface. In some embodiments, the exosome, microvesicle, or LNP is modified to contain one or more polypeptides. The levels of any desired surface marker can be modified directly on the exosome, microvesicle, or LNP (e.g., by contacting the complex with recombinantly produced polypeptides to bring about insertion in or conjugation to the membrane of the complex).

### Compositions

Even still further provided is a composition (e.g., a pharmaceutical composition). The composition can comprise a modRNA CAR construct hereof. In certain embodiments, the composition comprises a first modRNA construct comprising a nucleotide sequence encoding a CAR and at least one k-turn, wherein the at least one k-turn is upstream of the nucleotide sequence encoding the CAR. The composition can further comprise a second modRNA construct comprising a L7Ae modRNA construct hereof. In certain embodiments, the composition comprises the any combination of a L7Ae modRNA construct hereof and a modRNA CAR construct hereof. In certain embodiments, the composition comprises any of the L7Ae modRNA constructs described herein and any of the modRNA CAR constructs described herein.

The composition can comprise (i) a first modRNA construct comprising a nucleotide sequence encoding a CAR and at least one k-turn (e.g., a modRNA CAR construct); and, optionally, (ii) a second modRNA construct comprising a nucleotide sequence encoding at least one archaeal ribosomal protein L7Ae and at least one neutrophil-specific mircoRNA recognition sequence (e.g., a L7Ae modRNA construct), wherein the at least one neutrophil-specific microRNA recognition sequence is downstream of the nucleotide sequence encoding L7Ae.

In certain embodiments, the at least one neutrophil-specific mircoRNA recognition sequence of the second modRNA construct comprises miR-233, miR-142, or a combination of miR-233 and miR142. In certain embodiments, the CAR encoded by the first modRNA construct comprises a human CD4 transmembrane domain and a human CD3ζ intracellular domain. In certain embodiments, the CAR encoded by the first modRNA construct comprises a human CD8 transmembrane domain, a human 41BB co-stimulatory domain, and a human CD3ζ intracellular domain. In certain embodiments, the CAR encoded by the first modRNA construct comprises a 36-amino acid GBM-targeting CLTX peptide or other brain tumor-targeting ligand (e.g., IL-13) or a scFv. In certain embodiments, the CAR encoded by the first modRNA construct comprises a ligand that targets PSMA.

In certain embodiments of the composition, the at least one neutrophil-specific miRNA recognition sequence of the second modRNA construct is or comprises SEQ ID NO: 3, SEQ ID NO: 4, SEQ ID NO: 5, SEQ ID NO: 6, SEQ ID NO: 7, or a functional variant of SEQ ID NO: 3, SEQ ID NO: 4, SEQ ID NO: 5, SEQ ID NO: 6, or SEQ ID NO: 7. The first modRNA construct of a composition can comprise SEQ ID NO: 8, SEQ ID NO: 9, or a functional variant of SEQ ID NO: 8 or SEQ ID NO: 9. The first modRNA construct of a composition can comprise SEQ ID NO: 10, SEQ ID NO: 11, SEQ ID NO: 12, or a functional variant of SEQ ID NO: 10, SEQ ID NO: 11, or SEQ ID NO: 12.

In certain embodiments of the composition, the first modRNA construct comprises SEQ ID NO: 1, SEQ ID NO: 2, or a functional variant of SEQ ID NO: 1 or SEQ ID NO: 2. The first modRNA construct can comprise: a first sequence comprising SEQ ID NO: 1, SEQ ID NO: 2, or a functional variant of SEQ ID NO: 1 or SEQ ID NO: 2; and a second sequence comprising SEQ ID NO: 10, SEQ ID NO: 11, SEQ ID NO: 12, or a functional variant of SEQ ID NO: 10, SEQ ID NO: 11, or SEQ ID NO: 12. In certain embodiments, the first sequence is upstream of the second sequence.

The CAR encoded by the composition can comprise a neutrophil-specific transmembrane domain. The CAR encoded by the composition can comprise a neutrophil-specific transmembrane domain. In certain embodiments, the neutrophil-specific transmembrane can be or comprise a TLR4 polypeptide, a TLR2 polypeptide, a MET polypeptide, a G-CSFR, a Myd88 polypeptide, a TRIF polypeptide, a Syk peptide, a CD40 polypeptide, CD32a, Dectin-1, a IL6R, an FCER1G polypeptide, a TLR7, or a CD16 transmembrane polypeptide, a CD8 polypeptide, a CD28 polypeptide, an OX40 polypeptide, an ICOS polypeptide, a CTLA-4 polypeptide, a PD-1 polypeptide, a LAG-3 polypeptide, a BTLA polypeptide, a NKG2D, Dectin-1, CD16, or other Fcγ receptors.

The CAR encoded by the composition can comprise a neutrophil-specific transmembrane and/or co-stimulatory domain. In certain embodiments, the neutrophil-specific co-stimulatory domain can optionally be or comprise a 41BB co-stimulatory domain or a CD28 polypeptide. Additionally or alternatively, the neutrophil-specific transmembrane domain can optionally be or comprise CD32a, CD4, NKG2D, Dectin-1, a CD32 polypeptide, an IL-6 receptor, or CD16. In certain embodiments, one or both of the first and second modRNA (where applicable) constructs are encapsulated within an exosome, microvesicle, or a nanoparticle (NP) (each a “carrier”) (e.g., to facilitate delivery thereof to a subject). In certain embodiments, the first modRNA constructs are encapsulated by a first carrier. In certain embodiments, the second modRNA constructs are encapsulated by a second carrier. In certain embodiments, the first modRNA constructs are encapsulated by a first type of carrier and the second modRNA constructs are encapsulated by a second type of carrier, wherein the first and second types of carriers are different from each other. In certain embodiments, the first modRNA constructs and the second modRNA constructs are both encapsulated by the same type of carrier or the same carrier. In certain embodiments, mixtures of both the first and second modRNA constructs can be encapsulated by (or loaded in) the same carrier.

The composition can further comprise a pharmaceutically acceptable carrier and/or diluent. The term “pharmaceutically acceptable” and grammatical variations thereof, as they refer to compositions, carriers, diluents, reagents, and the like, are used interchangeably and indicate that the materials can be administered to or upon a mammal without undue toxicity, irritation, allergic response, and/or the production of undesirable physiological effects, such as nausea, dizziness, gastric upset, and the like as is commensurate with a reasonable benefit/risk ratio. In other words, it is a material that is not biologically or otherwise undesirable—i.e., the material may be administered to an individual directly or via an exosome or LNP, for example, without causing any undesirable biological effects or interacting in a significantly deleterious manner with any of the other components of the pharmaceutical composition.

The term “pharmaceutically acceptable carrier” is art-recognized and refers to a pharmaceutically acceptable material, composition or vehicle, such as a liquid or solid filler, diluent, excipient, solvent or encapsulating material, involved in carrying or transporting a composition or component thereof. Each carrier must be “acceptable” in the sense of being compatible with the subject composition and its components and not injurious to the patient. Some examples of materials, which may serve as pharmaceutically acceptable carriers, include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethanol; (20) phosphate buffered solutions; and (21) other non-toxic, compatible substances employed in pharmaceutical formulations.

The choice of carrier will be determined in part by the particular modRNA constructs vector, and/or host cells expressing the CAR, as well as by the particular method used to administer the sequences of the modRNA constructs. Accordingly, there are a variety of suitable formulations of the pharmaceutical composition. For example, the pharmaceutical composition can contain preservatives. Suitable preservatives may include, for example, methylparaben, propylparaben, sodium benzoate, and benzalkonium chloride. A mixture of two or more preservatives optionally can be used. The preservative or mixtures thereof are typically present in an amount of about 0.0001% to about 2% by weight of the total composition.

The composition can also comprise one or more pharmaceutically acceptable carriers, diluents, and/or other pharmaceutically acceptable components. The carriers, diluents, and/or other components can be determined in part by the particular route of administration (see, e.g., Remington's Pharmaceutical Sciences, 17th ed. (1985)). The ingredients of the composition can be of sufficiently high purity and sufficiently low toxicity such that the composition is suitable for administration to a human. The composition desirably is stable.

The particular formulation employed will also depend, at least in part, on the particular route of administration. For example, a formulation suitable for systemic, e.g., intravenous, administration, may differ from a formulation suitable for intracranial administration. Such modifications are within the ordinary skill in the art.

### Carriers

Also provided are carriers that encapsulate or otherwise comprise/carry one or more of the modRNA constructs described herein or any of the compositions described herein. The carriers can comprise an exosome, a microvesicle, or a NP. The exosomes, microvesicles, and NPs can comprise those described herein.

The exosome can have a diameter at or between about 30-500 nm (e.g., 30 nm to about 500 nm, about 30 nm to about 50 nm, about 30 nm to 500 nm, or 30-50 nm), at or between about 50-480 nm, at or about 70-460 nm, at or about 90-440 nm, at or about 110-420 nm, at or about 130-400 nm, at or about 130-400 nm, at or about 150-380 nm, at or about 170-360 nm, at or about 190-340 nm, at or about 210-320 nm, at or about 230-300 nm, or at or about 250-280 nm. In some embodiments, the exosome or NP comprises a membrane that forms a particle that has a diameter of 30-100 nm, 30-200 nm, or 30-500 nm. In some embodiments, the exosome comprises a membrane that forms a particle that has a diameter of 10-100 nm, 20-100 nm, 30-100 nm, 40-100 nm, 50-100 nm, 60-100 nm, 70-100 nm, 80-100 nm, 90-100 nm, 100-200 nm, 100-150 nm, 150-200 nm, 100-250 nm, 250-500 nm, or 10-1000 nm. In certain embodiments, the exosome has a diameter from about 30 nm to about 90 nm (such as, for example, 30-90 nm), from about 35 nm to about 85 nm (such as, for example, 35-85 nm), from about 40 nm to about 80 nm (such as, for example, 40-80 nm), from about 45 nm to about 75 nm (such as, for example, 45-75 nm), from about 50 nm to about 70 nm (such as, for example, 50-70 nm), or from about 55 nm to about 65 nm (such as, for example, 55-65 nm). The ranges set forth in this paragraph are inclusive of the stated end points and all 1 nm increments contained therein. In some embodiments, the membrane comprises lipids and fatty acids. In some embodiments, the membrane comprises one or more of phospholipids, glycolipids, fatty acids, sphingolipids, phosphoglycerides, sterols, cholesterols, and phosphatidylserine. In addition, the membrane can comprise one or more polypeptides and one or more polysaccharides, such as glycans.

In some embodiments, the exosome or NP is generated by a producer cell (or parental cell), such as, e.g., a mammalian cell. In some embodiments, the membrane of the exosome or NP comprises one or more molecules derived from the producer cell. The exosome can be generated in a cell culture system and isolated (e.g., by separating the exosome from the producer cell) using processes well known in the art. Separation can be achieved by sedimentation. For example, the exosome can have a specific density between 0.5-2.0, 0.6-1.0, 0.7-1.0, 0.8-1.0, 0.9-1.0, 1.0-1.1, 1.1-1.2, 1.2-1.3, 1.4-1.5, 1.0-1.5, 1.5-2.0, and 1.0-2.0 kg/m³. The stated ranges in this paragraph are inclusive of the stated end points and all 0.1 increments within the range.

In certain embodiments, the exosome or NP is synthetic. In some embodiments, the exosome or NP is modified (e.g., by introducing a payload or otherwise modifying the content of the complex, such as by changing the protein, lipid or glycan content of the membrane). For example, exosomes and NPs can be first isolated from a producer cell and then modified as desired, thereby generating synthetic exosomes or NPs, respectively. In some embodiments, the producer cell is modified. For example, an exogenous nucleic acid, an exogenous polypeptide or small molecule or toxin can be introduced into the producer cell. Alternatively or in addition, the producer cell can otherwise be modified (e.g., by modifying the cellular or membrane content, such as by changing the lipid or glycan content of the cell membrane). Exosomes and NPs generated from a modified producer cells comprise one or more of the modifications of the producer cell. The process produces synthetic exosomes. In some embodiments, both the producer cell and the exosome or NP isolated from the producer cell are modified as described herein. The term “isolated” means that the material is removed from its original environment, e.g., the natural environment if it is naturally occurring. For example, a naturally occurring neutrophil present within a living organism is not isolated, but the same neutrophil separated from some or all the coexisting materials in the natural system is isolated.

In some embodiments, the exosome, microvesicle, or NP facilitates delivery of the payload (i.e., the one or more modRNA constructs and/or compositions hereof) to a target (e.g., a neutrophil). The payload can comprise one or more of the modRNA constructs hereof and/or the compositions hereof, which can be internalized into the target (e.g., a neutrophil) that is contacted with the carrier.

Methods for loading carriers with the payload are described in U.S. Patent Application Publication Nos. 2014/0356382, 2020/0306297, and 2020/0347112. See also, Alvarez et al., Delivery of siRNA to the mouse brain by systemic injection of targeted exosomes, Nature Biotechnology 29:341-345 (2011).

In some embodiments, the carrier comprises polypeptides on its surface. In some embodiments, the carrier is modified to contain one or more polypeptides. The levels of any desired surface marker can be modified directly on the carrier (e.g., by contacting the complex with recombinantly produced polypeptides to bring about insertion in or conjugation to the membrane of the complex). For example, and without limitation, an exosome can be modified to comprise SEQ ID NO: 13 or SEQ ID NO: 14 on its surface. Völs et al., Targeted nanoparticles modify neutrophil function in vivo, Frontiers in Immunology 13 (2022).

In certain embodiments, especially where the carrier is a LNP, the carrier comprises one or more antibodies on its surface. For example, a LNP can comprise an anti-Ly6G antibody on its surface to target mouse neutrophils. Various different types of antibodies will be recognized as beneficial in this context and are known in the art with respect to targeting neutrophils.

In some embodiments, the producer cell is modified to contain the one or more polypeptides. In some embodiments, the producer cell naturally contains one or more polypeptides and exosomes derived therefrom also contain the polypeptides. Alternatively or in addition, the levels of any desired surface marker can be modified directly on the producer cell (e.g., by contacting the complex with recombinantly produced polypeptides to bring about insertion in or conjugation to the membrane of the cell). Alternatively, the producer cell can be modified by transducing an exogenous nucleic acid into the producer cell to express a desired surface marker. The surface marker can already be naturally present on the producer cell, in which case the exogenous construct can lead to overexpression of the marker and increased concentration of the marker in or on the producer cell. Alternatively, a naturally expressed surface marker can be removed from the producer cell (e.g., by inducing gene silencing in the producer cell). The polypeptides can confer different functionalities to the exosome (e.g., specific targeting capabilities, delivery functions (e.g., fusion molecules), enzymatic functions, increased or decreased half-life in vivo, etc).

Also provided are biocompatible nanoparticles (NPs) encapsulating or otherwise comprising/carrying one or more of the modRNA constructs described herein or any of the compositions described herein. NPs have become a promising strategy for anti-cancer treatment. Compared to small-molecule drugs, nanoparticles appear to be gradually enriched in tumors and maintained for a longer period of time. Li et al., Anti-cancer nanomedicines: a revolution of tumor immunotherapy, Frontiers Immunology. Sec. Cancer Immunity & Immunotherapy 11 (2020). Clinical approvals for tumor therapy include organic materials, such as pegylated and non-pegylated liposomes and albumin. In tumor therapy, NPs are mainly used for drug delivery, photothermal therapy, modification and preparation of engineered cells, imaging diagnosis, and lymph node tracings. The efficacy of existing anti-tumor agents can be improved when loaded into NPs. NPs, themselves, can trigger immunogenic tumor cell death and elicit both innate and adaptive immune responses for tumor control and prevention of metastasis. Ma et al., Near-infrared II phototherapy induces deep tissue immunogenic cell death and potentiates cancer immunotherapy, ACS Nano 13 (10): 11967-11980 (2019).

The NPs can comprise an LNP. The LNP can be any of those described above. The NPs can comprise cyclodextrin NPs, liposome NPs, polymeric NPs, solid lipid NPs, exosome NPs, autophagosome NPs, virus-like NPs, tumor lysate NPs, and/or gold NPs. The LNPs can comprise, for example, rough silica nanoparticles, cytosine arabinoside-based liposomes (e.g., DepoCyt®), polyamidoamine (PAMAM) dendrimer-albumin nanoparticles, and fullerene (e.g., gadofullerenol/fullerenol). The rough silica nanoparticles can be biodegradable mesoporous organic silica.

### Methods and Uses

Methods of producing CAR-expressing neutrophils in a subject (e.g., in vivo) are provided. In certain embodiments, a method of producing CAR-expressing neutrophils in a subject comprises administering to the subject at least one modified RNA (modRNA) constructs, the at least two modRNA constructs comprising a modRNA construct comprising a nucleotide sequence encoding a CAR and at least one k-turn, wherein the at least one k-turn is upstream of the nucleotide sequence encoding CAR. The first modRNA construct can comprise any of the CAR modRNA constructs described herein. When administered to a subject, the at least one modRNA construct can be endocytosed by a neutrophil in the subject, and the neutrophil can express the CAR.

The at least one modRNA construct can comprise the first modRNA construct and a second modRNA construct. The second modRNA construct can comprise a nucleotide sequence encoding the archaeal ribosomal protein L7Ae and at least one neutrophil-specific microRNA recognition sequence. In certain embodiments, the at least one neutrophil-specific microRNA recognition sequence is downstream of the nucleotide sequence encoding L7Ae.

The second modRNA construct can comprise any of the L7Ae modRNA constructs described herein. In certain embodiments, when administered to a subject, the two modRNA constructs can be endocytosed by a neutrophil in the subject such that the neutrophil expresses the CAR.

The subject can be a mammal. The subject can be a human. The subject can be a mouse. The subject can be a canine.

In certain embodiments, the subject is a human and the at least one neutrophil-specific microRNA recognition sequence of the second modRNA construct is or comprises miR-233, miR-142, or both miR-233 and miR-142 or a functional variant thereof. In certain embodiments, the subject is a human and the at least one neutrophil-specific miR recognition sequence of the first modRNA constrict is or comprises SEQ ID NO: 3, SEQ ID NO: 4, SEQ ID NO: 5, SEQ ID NO: 6, SEQ ID NO: 7, or a functional variant of SEQ ID NO: 3, SEQ ID NO: 4, SEQ ID NO: 5, SEQ ID NO: 6, or SEQ ID NO: 7. In certain embodiments, the subject is a human and the at least one neutrophil-specific miR recognition sequence of the first modRNA constrict is or comprises SEQ ID NO: 3, SEQ ID NO: 5, both SEQ ID NO: 3 and SEQ ID NO: 5, a functional variant of SEQ ID NO: 3 or SEQ ID NO: 5, or functional variants of one or both of SEQ ID NO: 3 and SEQ ID NO: 5.

The CAR encoded by the first modRNA construct can comprise a neutrophil-specific transmembrane domain. The neutrophil-specific transmembrane domain can be or comprise a TLR4 polypeptide, a TLR2 polypeptide, a MET polypeptide, a G-CSFR, a Myd88 polypeptide, a TRIF polypeptide, a Syk peptide, a CD40 polypeptide, CD32a, Dectin-1, a IL6R, an FCER1G polypeptide, a TLR7, or a CD16 transmembrane polypeptide, a CD8 polypeptide, a CD28 polypeptide, an OX40 polypeptide, an ICOS polypeptide, a CTLA-4 polypeptide, a PD-1 polypeptide, a LAG-3 polypeptide, a BTLA polypeptide, a NKG2D, Dectin-1, CD16, or other Fcγ receptors. In certain embodiments of the method, the CAR encoded by the first modRNA construct comprises a neutrophil-specific transmembrane and/or co-stimulatory domain, wherein the neutrophil-specific co-stimulatory domain can optionally be or comprise a 41BB co-stimulatory domain or a CD28 polypeptide and the neutrophil-specific transmembrane domain can optionally be or comprise CD32a, CD4, NKG2D, Dectin-1, a CD32 polypeptide, an IL-6 receptor, or CD16.

In certain embodiments of the method of producing CAR-expressing neutrophils in a subject, the subject is human and the CAR encoded by the first modRNA construct comprises a human CD4 transmembrane domain and a human CD3ζ intracellular domain. In certain embodiments of the method of producing CAR-expressing neutrophils in a subject, the subject is human and the CAR encoded by the first modRNA construct comprises a human CD8 transmembrane domain, a human 41BB co-stimulatory domain, and a human CD3ζ intracellular domain. In certain embodiments of the method of producing CAR-expressing neutrophils in a subject, the subject is human and the CAR comprises a 36-amino acid GBM-targeting CLTX peptide or other brain tumor-targeting ligand (e.g., IL-13) or a scFv. In certain embodiments of the method of producing CAR-expressing neutrophils in a subject, the subject is human and the CAR comprises a ligand that targets PSMA.

In certain embodiments, the at least one modRNA construct is administered in a carrier. The carrier can be any of the carriers described herein. In certain embodiments, the carrier is an exosome. The exosome can be or comprise any of the exosomes described herein. The exosome can be administered systemically.

In certain embodiments, the at least one modRNA construct is administered in a NP. The NP can be or comprise any of the LNPs or NPs described herein. The LNP can be administered systemically.

In certain embodiments, the carrier is a microvesicle and, optionally, the microvesicle can be administered systemically.

In certain embodiments, the amount of modRNA constructs administered should be sufficient to result in phagocytosis of cancerous cells by neutrophils, such that the progression of cancer (e.g., the proliferation of cancerous cells and/or the metastasis thereof) is inhibited and desirably eradicated. Phagocytosis of cancerous cells by neutrophils can be achieved at a low level of expression of modRNA in neutrophils, such as expression in about 2% of the neutrophils. Expression in about 2% to about 8% of neutrophils can be achieved when the modRNA is contained within carriers. Expression in 2-4% neutrophils can be achieved when the modRNA is contained within NPs. Expression in 2-8% neutrophils can be achieved when the modRNA is contained within exosomes. Expression in 2-8% neutrophils can be achieved when the modRNA is contained within microvesicles.

The amount of modRNA to be administered to achieve expression in at least about 2% of the neutrophils is at least about 5 μg/20 g of body weight. The exact amount of modRNA constructs required can vary from one subject to the next, depending on factors such as the patient's general health, age, and state/severity of the cancer.

The modRNA, such as modRNA in carriers, can be administered by any suitable route. Such routes include, but are not limited to, systemic routes, such as intravenous administration. The formulation of compositions suitable for administration of modRNA, including compositions suitable for intravenous administration, is within the ordinary skill in the art.

In certain embodiments, the method of producing CAR-expressing neutrophils in a subject further comprises administering a drug to the subject. In certain embodiments, the method further comprises administering a therapeutically effective amount of a drug to the subject. In certain embodiments, the drug is a nanodrug. In certain embodiments, the nanodrug is a chemotherapeutic agent.

The drug can comprise any drug (including, without limitation, a nanodrug) or prodrug that can be used for therapeutic or prophylactic treatment, such as the therapeutic treatment of cancer (e.g., a chemotherapeutic agent). The drug can be a preclinical or clinical nanodrug or prodrug, an antineoplastic/chemotherapeutic drug, or a radiosensitizer. Antineoplastic/chemotherapeutic drugs can be categorized as alkylating agents, antimetabolites, natural products, hormones and antagonists, and miscellaneous (see, e.g., Antineoplastic Agents, www[dot]ncbi[dot]nlm[dot]nih[dot]gov/books/NBK548022/). Such drugs also can be classified by indication, mechanism of action, chemical structure, or cytotoxic/nonspecific vs. noncytotoxic/targeted. Examples of alkylating agents include, but are not limited to, altretamine, bendamustine, busulfan, carmustine, chlorambucil, cyclophosphamide, dacarbazine, ifosfamide, lomustine, mechlorethamine, melphalan, procarbazine, streptozocin, temozolomide, thiotepa, trabectedin, and platinum coordination complexes (e.g., carboplatin, cisplatin (a radiosensitizer), and oxaliplatin). Examples of antibiotics and cytotoxic agents include, but are not limited to, bleomycin, catinomycin, daunorubicin, doxorubicin, epirubicin, idarubicin, mitomycin (a radiosensitizer), mitoxantrone, plicamycin, and valrubicin. Nonlimiting examples of antimetabolites include antifolates (e.g., methotrexate, pemetrexed, pralatrexate, and trimetrexate), purine analogues (e.g., azathioprine, cladribine, fludarabine (a radiosensitizer), mercaptopurine, and thioguanine), and pyrimidine analogues (e.g., azacytidine, capecitabine, cytarabine, decitabine, floxuridine, fluorouracil (a radiosensitizer), gemcitabine (a radiosensitizer), and trifluridine/tipriacil). Biologic response modifiers include aldesleukin (IL-2), denileukin diftitox, and interferon gamma (IFNγ) as examples. Histone deactylase inhibitors include belinostat, Panobinostat, romidepsin, and vorinostat as examples. Hormonal agents include anti-androgens (e.g., abiraterone, apalutamide, bicalutamide, cyproterone, enzalutamide, flutamide, and nilutamide), anti-estrogens and aromatase inhibitors (e.g., anastrozole, exemestane, fulvestrant, letrozole, raloxifene, tamoxifen, and toremifene), gonadotropin releasing hormone analogues (e.g., degarelix, goserelin, histrelin, leuprolide, and triptorelin), and peptide hormones (e.g., lanreotide, octreotide, and pasireotide). Examples of monoclonal antibodies are numerous and include alemtuzumab, atezolizumab, bevacizumab, blinatumomab, cemiplimab, cetuximab, daratumumab, dinutuximab, elotuzumab, gemtuzumab, and inotuzumab among others. Likewise, examples of protein kinase inhibitors are numerous and include abemaciclib, acalabrutinib, binimetinib, bortezomib, cabozantinib, carfilzomib, dabrafenib, dacomitinib, enasidenib, encorafenib, fedratinib, gefitinib, ibrutinib, lapatinib, midostaurin, and neratinib among others. Taxanes include, but are not limited to, cabazitaxel, docetaxel (a radiosensitizer), and paclitaxel (a radiosensitizer). Topoisomerase inhibitors include, but are not limited to, etoposide, irinotecan teniposide (a radiosensitizer), and topotecan. Vinca alkaloids include, but are not limited to, vinblastine, vincristine, and vinorelbine. Other antineoplastic/chemotherapeutic agents include asparaginase, bexarotene, eribulin, everolimus, hydroxyurea (a radiosensitizer), ixabepilone, lenalidomide, mitotane, omacetaxine, pomalidomide, tagraxofusp, telotristate, temsirolimus, thalidomide, and venetoclax. In various embodiments, the drug is tirapazamine ((TPZ); an example of a benzotriazine-di-N-oxide hypoxic cytotoxin, which can be used as a radiosensitizer), temozolomide (an example of an alkylating agent), climacostol, or indole-3-acetic acid. In other embodiments, the drug is everolimus, bevacizumab, belzutifan, carmustine, naxitamab-gqgk, or lomustine.

In embodiments in which the drug comprises a prodrug, the prodrug can be activated by hypoxic conditions, acidic pH, an enzyme (e.g., horseradish peroxidase), irradiation, and the like.

The subject can have cancer. As used herein, “cancer” includes any neoplastic condition, whether malignant, pre-malignant or non-malignant. Generally, however, the neoplastic condition is malignant. Both solid and non-solid tumors are encompassed, and “cancer(ous) cell” is used interchangeably with “tumor(ous) cell” unless stated otherwise.

Examples of cancers include, but are not limited to, leukemia (e.g., ALL, AML, CLL, and CML), adrenocortical carcinoma, AIDS-related cancer (e.g., Kaposi sarcoma), lymphoma (e.g., T-cell, Hodgkins, and non-Hodgkins), astrocytoma, basal cell carcinoma, bladder cancer, bone cancer, brain cancer (e.g., glioblastoma (GBM)), breast cancer, prostate cancer, lung cancer, cervical cancer, colon cancer, colorectal cancer, DCIS, esophageal cancer, gastric cancer, glioma, head and neck cancer, liver cancer, stomach cancer, pancreatic cancer, kidney cancer (e.g., renal cell and Wilms), oral cancer, oropharyngeal cancer, ovarian cancer, testicular cancer, and throat cancer. In various embodiments, the cancer is a brain cancer, such as GBM. In various other embodiments, the cancer is a prostate cancer.

A method of producing CAR-expressing neutrophils ex vivo for administration to a subject is also provided. In certain embodiments, the method of producing CAR-expressing neutrophils ex vivo for administration to a subject comprises (i) isolating neutrophils from a subject, (ii) contacting the isolated neutrophils with at least one modRNA construct, the at least one modRNA construct comprising a first modRNA construct comprising a nucleotide sequence encoding a CAR and at least one k-turn, wherein the at least one k-turn is upstream of the nucleotide sequence encoding CAR, whereupon when the modRNA is endocytosed by the isolated neutrophils, the neutrophils express the CAR, and (iii) administering the CAR-expressing neutrophils to the subject. In certain embodiments, the at least one modRNA construct can comprise: (a) the first modRNA construct comprising a nucleotide sequence encoding a CAR and at least one k-turn, wherein the at least one k-turn is upstream of the nucleotide sequence encoding CAR; and (b) a second modRNA construct comprising a nucleotide sequence encoding the archaeal ribosomal protein L7Ae and at least one neutrophil-specific microRNA recognition sequence, wherein the at least one neutrophil-specific microRNA recognition sequence is downstream of the nucleotide sequence encoding L7Ae. The first modRNA construct can comprise any of the modRNA CAR constructs described herein. The second modRNA construct can comprise any of the L7Ae modRNA constructs described herein.

The subject can comprise any of the subjects described herein. The subject can be a human, and the second modRNA construct can comprise at least one neutrophil-specific microRNA recognition sequence selected from miR-233, miR-142, and a combination of both miR-233 and miR-142. The subject can be a human, and the CAR can comprise a human CD4 transmembrane domain and a human CD3ζ intracellular domain. The subject can be a human, and the CAR can comprise a human CD8 transmembrane domain, a human 41BB co-stimulatory domain, and a human CD3ζ intracellular domain. The CAR can comprise a 36-amino acid GBM-targeting chlorotoxin peptide or other brain tumor-targeting ligand (e.g., IL-13) or a scFv. The CAR can comprise a ligand that targets PSMA. The CAR can comprise a ligand that targets tumors or other brain disorders (e.g., Alzheimer's disease). The CAR can comprise a ligand that targets autoimmune diseases (e.g., systemic lupus erythematosus or the like), fibrosis (e.g., cardiac, lung, or liver fibrosis), and the like. It will be recognized that the CAR can comprise a targeting ligand known in the art to target delivery to a specific disease or disorder in the subject.

The method can further comprise administering to the subject a therapeutically effective amount of a drug (e.g., a nanodrug). The drug can be any of the drugs described herein. In certain embodiments, the drug is a nanodrug comprising a chemotherapeutic agent. The subject can have cancer. The cancer can be a brain cancer. The brain cancer can be glioblastoma. The cancer can be prostate cancer.

A method of treating cancer in a subject (e.g., in need thereof) is also provided. The method of treating cancer in a subject can comprise administering to a subject: a first modRNA construct comprising a nucleotide sequence encoding a CAR and at least one k-turn, wherein the at least one k-turn is upstream of the nucleotide sequence encoding CAR.

The first modRNA construct can comprise any of the CAR modRNA constructs described herein.

In certain embodiments, the method can further comprise administering to a subject a second modRNA construct comprising a nucleotide sequence encoding the archaeal ribosomal protein L7Ae and at least one neutrophil-specific microRNA recognition sequence, wherein the at least one neutrophil-specific microRNA recognition sequence is downstream of the nucleotide sequence encoding L7Ae. The second modRNA construct can comprise any of the L7Ae modRNA constructs described herein.

When administered to a subject, the at least one modRNA construct can be endocytosed by a neutrophil in the subject such that the neutrophil expresses the CAR. The first and/or second modRNA constructs can be formulated into a composition which can be administered to the subject. The composition can comprise any of the compositions described herein.

The subject can be a mammal. The subject can be a human. The subject can be a mouse. The subject can be a canine.

In certain embodiments of both the method of treating cancer and the method of producing CAR-expressing neutrophils in a subject (e.g. in vivo) described herein, the method can further comprise administering to the subject a second therapy (e.g., using a therapeutically effective amount) and provides an increased cytotoxic effect on a cancer in the subject as compared to administration to the subject of the combination of modRNA constructs alone.

The second therapy can comprise surgical removal of one or more cancerous cells from the subject, chemotherapy, imaging (e.g., a cancer site in the subject), and/or radiotherapy (e.g., a therapeutically effective amount thereof). In certain embodiments, the method further comprises administering to the subject a therapeutically effective amount of chemotherapy. In certain embodiments, the method further comprises administering to the subject a therapeutically effective amount of radiotherapy. In certain embodiments, the method further comprises administering to the subject a therapeutically effective amount of both chemotherapy and radiotherapy.

In certain embodiments, the method further comprises imaging the subject (e.g., a cancer site in the subject). The cancer can be imaged prior to administration to the subject of the modRNA constructs hereof and/or the compositions hereof. A cancer, additionally, or alternatively, can be imaged during or after administration to assess metastasis, for example, and the efficacy of treatment. In some embodiments, imaging occurs by positron emission tomography (PET) imaging, magnetic resonance imaging (MRI), or single-photon-emission computed tomography (SPECT)/computed tomography (CT) imaging. The imaging method can be any suitable imaging method known in the art.

In some embodiments, the method further comprises imaging a solid tumor cancer prior to or during administration of the combinations of modRNA constructs hereof, a composition comprising a combination of modRNA constructs hereof, and/or the second therapy.

The terms “treat,” “treating,” “treated,” and “treatment” (with respect to a disease or condition, such as cancer) are used to describe a method for obtaining beneficial or desired results, such as clinical results, which can include, but are not limited to, one or more of improving a condition associated with a disease, curing a disease, lessening severity of a disease, increasing the quality of life of one suffering from a disease, prolonging survival and/or a prophylactic treatment. In reference to cancer, in particular, the terms “treat,” “treating,” “treated,” or “treatment” can additionally mean reducing the size of a tumor, completely or partially removing the tumor (e.g., a complete or partial response), stabilizing a disease, preventing progression of the cancer (e.g., progression-free survival), or any other effect on the cancer that would be considered by a physician to be a therapeutic or prophylactic treatment of the cancer. More particularly, curative treatment refers to any of the alleviation, amelioration and/or elimination, reduction and/or stabilization (e.g., failure to progress to more advanced stages) of a sign/symptom, as well as delay in progression of a sign/symptom of a particular disorder. Prophylactic treatment refers to any of the following: halting the onset, reducing the risk of development, reducing the incidence, delaying the onset, reducing the development, and increasing the time to onset of symptoms of a particular disorder. Desirable effects of treatment can include, but are not limited to, preventing occurrence or recurrence of a disease, alleviation of symptoms, diminishment of any direct or indirect pathological consequences of the disease, preventing metastasis, decreasing the rate of disease progression, amelioration or palliation of the disease state, and remission or improved prognosis. In some embodiments, compositions are used to delay development of a disease and/or tumor, or to slow (or even halt) the progression of a disease and/or tumor growth.

The term “patient” or “subject” includes human and non-human animals, such as companion animals (dogs and cats and the like) and livestock animals. Livestock animals are animals raised for food production. The subject to be treated is preferably a mammal, in particular a human being.

As used herein, the term “administering” includes all means of introducing the neutrophils and pharmaceutical compositions comprising same, to the patient. Examples include, but are not limited to, oral (po), parenteral, systemic/intravenous (iv), intramuscular (im), subcutaneous (sc), transdermal, intrasternal, intraarterial, intraperitoneal, epidural, intraurethral, intranasal, buccal, ocular, sublingual, vaginal, rectal, and the like. Routes of administration to the brain include, but are not limited to, intraparenchymal, intraventricular, intracranial, and the like.

Illustrative means of parenteral administration include needle (including microneedle) injectors, needle-free injectors and infusion techniques, as well as any other means of parenteral administration recognized in the art. Parenteral formulations are typically aqueous solutions, which may contain excipients, such as salts, carbohydrates and buffering agents (preferably at a pH in the range from about 3 to about 9). The preparation of parenteral formulations under sterile conditions may readily be accomplished using standard pharmaceutical techniques well-known to those skilled in the art.

The modRNA constructs and/or CAR-expressing neutrophils can be formulated as pharmaceutical compositions and administered to a mammalian host, such as a human patient, in a variety of forms adapted to the chosen route of administration. For example, the pharmaceutical composition can be formulated for and administered via oral or parenteral, intravenous, intraarterial, intraperitoneal, intrathecal, epidural, intracerebroventricular, intraurethral, intrasternal, intracranial, intratumoral, intramuscular, topical, inhalation and/or subcutaneous routes. Indeed, the neutrophils, or composition comprising the same, can be administered directly into the blood stream, into muscle, or into an internal organ.

The modRNA constructs and/or CAR-expressing neutrophils and/or compositions can be administered via infusion or injection (e.g., using needle (including microneedle) injectors and/or needle-free injectors). Solutions of the composition can be aqueous, optionally mixed with a nontoxic surfactant and/or can contain carriers or excipients such as salts, carbohydrates and buffering agents (preferably at a pH of from 3 to 9).

The percentage of the modRNA constructs and/or CAR-expressing neutrophils and/or compositions and preparations may vary and may be between about 1 to about 99% weight of the active ingredient(s) and a binder, excipients, a disintegrating agent, a lubricant, and/or a sweetening agent (as are known in the art). The amount of the modRNA constructs and/or CAR-expressing neutrophils in such therapeutically useful compositions is such that an effective dosage level will be obtained.

In some embodiments, the modRNA constructs and/or CAR-expressing neutrophils are administered as a composition comprising one or more pharmaceutically acceptable carriers, adjuvants, diluents, excipients, vehicles, or a combination of any of the foregoing.

The term “therapeutically effective amount” as used herein, refers to that amount of engineered neutrophils that elicits the biological or medicinal response in a tissue system, animal or human that is being sought by a researcher, veterinarian, medical doctor or other clinician (e.g., a desired therapeutic effect), which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the modRNA construct combinations and engineered neutrophils may be decided by the attending physician within the scope of sound medical judgment. In the treatment of cancer, a desired therapeutic effect can range from inhibiting the progression of cancer, e.g., proliferation of cancerous cells and/or the metastasis thereof. Desirably, the administration of a therapeutically sufficient amount kills cancerous cells, such that the number of cancerous cells decreases, desirably to the point of eradication.

The specific therapeutically effective dose level of modRNA constructs or CAR neutrophils for any particular patient will depend upon a variety of factors, including the disorder being treated and the state/severity of the disorder; the specific composition employed; the age, body weight, general health, gender and diet of the patient; the time and route of administration; the duration of the treatment; drugs used in combination or coincidentally with the engineered neutrophils; and like factors well-known to the researcher, veterinarian, medical doctor or other clinician of ordinary skill.

Depending upon the route of administration, a wide range of permissible dosages are contemplated herein. The dosages may be single or divided and may administered according to a wide variety of protocols, including q.d. (once a day), b.i.d. (twice a day), t.i.d. (three times a day), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

Multiple infusions may be required in order to treat a subject effectively. For example, 2, 3, 4, 5, 6 or more separate infusions may be administered to a subject at intervals of from about 24 hours to about 48 hours, or every 3, 4, 5, 6, or 7 days. Infusions may be administered weekly, biweekly, or monthly. Monthly administrations can be repeated from 2-6 months or longer, such as 9 months to year.

Use of at least one modRNA construct in the preparation of a medicament for treating a cancer is also provided. In certain embodiments, the use comprises using at least one modRNA construct in the preparation of a medicament, the at least one modRNA construct comprising a first modRNA construct comprising a nucleotide sequence encoding a CAR and at least one k-turn.

The first modRNA construct can comprise any of the modRNA CAR constructs described herein. For example, the CAR encoded by the first modRNA construct can comprise a human CD4 transmembrane domain and a human CD3ζ intracellular domain. The CAR can comprise a human CD8 transmembrane domain, a human 41BB co-stimulatory domain, and a human CD3ζ intracellular domain. The CAR can comprise a 36-amino acid GBM-targeting chlorotoxin peptide or other brain tumor-targeting ligand or a scFv. The CAR can comprise a ligand that targets PSMA.

The at least one modRNA constructs can comprise a combination of the first modRNA construct (i.e., any of the modRNA CAR constructs hereof) and a second modRNA construct. The second modRNA construct can comprise a nucleotide sequence encoding an L7Ae and at least one neutrophil-specific microRNA recognition sequence. The at least one neutrophil-specific microRNA recognition sequence can be downstream of the nucleotide sequence encoding L7Ae. The second modRNA construct can be any of the L7Ae modRNA constructs described herein. The second modRNA construct can comprise at least one neutrophil-specific microRNA recognition sequence that is or comprises miR-233, miR-142, or both miR-233 and miR-142.

The cancer can be a brain cancer. The cancer can be glioblastoma. The cancer can be a prostate cancer.

In certain embodiments, the at least one modRNA constructs can be encapsulated within a carrier (e.g., an exosome, a NP, or a microvesicle). In certain embodiments, the medicament can be formulated for systemic administration. The at least one modRNA constructs can be encapsulated within a NP, a microvesicle, or an exosome. The medicament can be formulated for administration in combination with a therapeutically effective amount of a drug, optionally a nanodrug.

### General

All patents, patent application publications, journal articles, textbooks, and other publications mentioned in the specification are indicative of the level of skill of those in the art to which the disclosure pertains.

In the above description, numerous specific details are set forth to provide a thorough understanding of the present disclosure. Particular examples may be implemented without some or all of these specific details and it is to be understood that this disclosure is not limited to particular biological systems, particular cancers, or particular organs or tissues, which can, of course, vary but remain applicable in view of the data provided herein.

Additionally, various techniques and mechanisms of the present disclosure sometimes describe a connection or link between two components. Words such as attached, linked, coupled, connected, and similar terms with their inflectional morphemes are used interchangeably, unless the difference is noted or made otherwise clear from the context. These words and expressions do not necessarily signify direct connections but include connections through mediate components. It should be noted that a connection between two components does not necessarily mean a direct, unimpeded connection, as a variety of other components may reside between the two components of note. Consequently, a connection does not necessarily mean a direct, unimpeded connection unless otherwise noted.

Further, will be understood that the disclosure is presented in this manner merely for explanatory purposes and the principles and embodiments described herein may be applied to compounds and/or composition components that have configurations other than as specifically described herein. Indeed, it is expressly contemplated that the components of the composition and compounds of the present disclosure may be tailored in furtherance of the desired application thereof.

Unless defined otherwise, all technical and scientific terms used herein have the same meaning as commonly understood by one of skill in the chemical and biological arts. Although any methods and materials similar to or equivalent to those described herein can be used in the practice or testing of the subject of the present application, the preferred methods and materials are described herein.

The term “about,” when referring to a number or a numerical value or range (including, for example, whole numbers, fractions, and percentages), means that the number or numerical range referred to is an approximation within experimental variability (or within statistical experimental error), and thus the numerical value or range can vary between 1% and 15% of the stated number or numerical range (e.g., +/−5% to 15% of the recited value), provided that one of ordinary skill in the art would consider equivalent to the recited value (e.g., having the same function or result).

When ranges are used herein for physical properties, such as molecular weight, or chemical properties, such as chemical formulae, all combinations and sub-combinations of ranges and specific embodiments therein are intended to be included.

The disclosure may be suitably practiced in the absence of any element(s) or limitation(s), which is/are not specifically disclosed herein. Thus, for example, each instance herein of any of the terms “comprising,” “consisting essentially of,” and “consisting of” (and related terms such as “comprise” or “comprises” or “having” or “including”) can be replaced with the other mentioned terms. Likewise, the singular forms “a,” “an,” and “the” include plural references unless the context clearly dictates otherwise. Thus, for example, references to “the method” include one or more methods and/or steps of the type, which are described and/or which will become apparent to those ordinarily skilled in the art upon reading the disclosure. The term “substantially” can allow for a degree of variability in a value or range, for example, within 90%, within 95%, or within 99% of a stated value or of a stated limit of a range.

It is recognized that various modifications are possible within the scope of the disclosure. Thus, although the present disclosure has been specifically disclosed in the context of preferred embodiments and optional features, those skilled in the art may resort to modifications and variations of the concepts disclosed herein. Such modifications and variations are considered to be within the scope of the disclosure as claimed herein.

It is therefore intended that this description and the appended claims will encompass all modifications and changes apparent to those of ordinary skill in the art based on this disclosure. For example, where a method of treatment or therapy comprises administering more than one treatment, compound, or composition to a subject, it will be understood that the order, timing, number, concentration, and volume of the administration is limited only by the medical requirements and limitations of the treatment (i.e., two treatments can be administered to the subject, e.g., simultaneously, consecutively, sequentially, alternatively, or according to any other regimen).

Additionally, in describing representative embodiments, the disclosure may have presented a method and/or process as a particular sequence of steps. To the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences may be varied and still remain within the spirit and scope of the present disclosure.

Further, the use of headings and subheadings is for ease of reference, given the length of the document. Description under one heading or subheading (such as a subheading in the Detailed Description) is not intended to be limited to only the subject matter set forth under that particular heading or subheading.

### EXAMPLES

The following examples serve to illustrate the present disclosure. The examples are not intended to limit the scope of the claimed invention in any way.

### Example 1

### Synthesis of Modified mRNA (modRNA)

All L7Ae, k-turn tdTomato and chlorotoxin (CLTX)-CAR template DNA was polymerase chain reaction (PCR) amplified from the plasmids according to protocols described in Haideri et al., Robust genome editing via modRNA-based Cas9 or base editor in human pluripotent stem cells, Cell Reports Methods 2 (9): 100290 (2022). Briefly, the PCR product was run on a 1% agarose gel, the band at the appropriate size was excised, and the DNA was extracted using the Zymoclean Gel DNA Recovery kit (Zymo Research Corp., Irvine, CA). Purified insert DNA was cloned into a linearized modRNAc1 plasmid using NEBuilder HiFi Kit (New England Biolabs, Ipswich, MA). The DNA template for modRNA synthesis was PCR-amplified from the successfully cloned modRNAc1 plasmid followed by PCR purification using DNA Clean & Concentrator-5 (Zymo Research Corp., Irvine, CA). ModRNA was synthesized from the PCR DNA template via in vitro transcription (IVT) using the MEGAscript T7 Transcription kit (Thermo Fisher Scientific Inc., Waltham, MA) supplemented with 8.1 mM ATP, 2.7 mM guanosine triphosphate (GTP), 8.1 mM cytidine 5′-triphosphate (CTP), 2.7 mM N1-methyl-pseudo-urindine triphosphate (UTP) (TriLink Biotechnologies, San Diego, CA), and 10 mM Anti-Reverse Cap Analog (ARCA) (TriLink Biotechnologies, San Diego, CA).

The IVT reaction product was treated with DNase I to remove the DNA template and then purified using the MEGAclear transcription clean-up kit (Thermo Fisher Scientific, Waltham, MA). RNA concentration was measured using a NanoDrop (Thermo Fisher Scientific, Waltham, MA).

### Example 2

### Delivery of modRNA into Neutrophils In Vitro Using Lipid Nanoparticles and Exosomes

Commercially available lipid nanoparticles (LNPs) and exosomes were tested for efficiency of transfer of green fluorescent protein (GFP) modRNA into neutrophils in vitro. See FIGS. 2A-2B.

Synthetic L7Ae: k-turn RNP switch, where L7Ae is an archaeal ribosomal protein, has been reported to regulate the translation of a designed mRNA in mammalian cells. Saito et al., Synthetic translational regulation by an L7Ae-kink-turn RNP switch, Nature Chemical Biology 6:71-78 (2010); Wroblewska et al. (2015), supra.

A neutrophil-specific L7Ae: k-turn RNP switch was designed by incorporating neutrophil-specific microRNA 223 (miR223) and microRNA 142 (miR142) recognition sites into a L7Ae modRNA construct (FIG. 2C). The target modRNA tdTomato or modRNA CAR constructs included two k-turns that are recognized by L7Ae, which prevented the translation of tdTomato or CAR. In the presence of miR223 and/or miR142, which are expressed in human and mouse neutrophils, translation of L7Ae was stopped, resulting in neutrophil-specific expression of modRNA tdTomato or CAR (FIG. 2D).

The transmembrane (tm), intracellular co-stimulatory (intra) and signaling domains were optimized using (i) human CD4-tm and CD3ζ, (ii) human CD8-tm, 41BB, and CD3ζ, or (iii) mouse CD28-tm, CD28-intra and CD3ζ, respectively (FIG. 2E). Exosome-mediated delivery of human CAR #2 and mouse CAR #3 into primary human and mouse neutrophils were the most efficient in killing glioblastoma (GBM) cells in vitro (FIGS. 2F-2G). Co-delivery of modRNA constructs and nanodrugs could further boost the antitumor cytotoxicity of primary neutrophils against tumor cells (FIG. 2H).

### Example 3

### Distribution and In Vivo Engineering of CAR-Neutrophils

The feasibility of using optimized exosome delivery and the L7Ae switch system to engineer CAR-neutrophils directly in vivo was tested. Firstly, the efficiency and distribution of in vivo engineered CAR-neutrophils in wild-type mice 24 hours after systemic administration of eGFP modRNA was determined. Consistent with the in vitro data, exosome-mediated modRNA delivery into primary neutrophils was more efficient (FIG. 2I). About 2-4% and 2-8% neutrophils, isolated from blood, bone marrow, liver and spleen, expressed eGFP 24 hours after LNP and exosome-mediated modRNA delivery, respectively. Since a normal mouse has ~50 million and 2.3 million neutrophils in bone marrow and blood, respectively, a total of more than 2.7 million neutrophils expressed eGFP after modRNA delivery in a single mouse. Boxio et al. (2004), supra.

### Example 4

### Xenograft Studies in Immunodeficient Mice

Immunodeficient NOD.Cg-RAG^{1tm1Mom} IL2rg^{tm1Wjl}/SzJ (NRG) mice were bred and maintained. In situ xenograft murine models were constructed via intracranial injection of 5×10⁵ luciferase-expressing GBM cells into the brain of immunodeficient mice. Neutrophils (5×10⁶) were intravenously injected into these mice at day 4, day 11, day 18, and day 25, and blood samples were collected from these mice at day 5, day 12, day 19, and day 26. Tumor burden was monitored by bioluminescence imaging (BLI) system (Spectral Ami Optical Imaging System, Spectral Instruments, Inc., Tucson, TZ), and body weights of experimental mice were measured once per week. Collected blood cells were stained with CD45 and analyzed in an Accuri C6 plus flow cytometer (Beckton Dickinson and Company, Franklin Lakes, NJ). Blood samples were also subjected to enzyme-linked immunosorbent assay (ELISA) to measure human TNFα and IL-6 cytokine release (Invitrogen, Thermo Fischer Scientific, Waltham, MA). At the end of treatment, tumors were collected for hematoxylin and eosin (H&E) staining.

For in vivo biodistribution analysis, fluorescence images were captured by the Spectral Ami Optical Imaging System at three and 24 hours after intravenous injection of Cy5 (Lumiprobe)-labeled neutrophils.

The in situ GBM xenograft model was then implemented for a proof-of-concept in vivo tumor-killing study, and CAR modRNA was systemically administered into tumor-bearing mice (FIG. 2J). As compared to phosphate-buffered saline (PBS) or the tdTomato modRNA control group, the group systemically administered CLTX-CAR modRNA exhibited significantly slower tumor growth in mouse brain and extended animal survival (FIGS. 2K-2L). This data supports the feasibility and translational potential of using modRNA to engineer CAR-neutrophils directly in vivo for targeted cancer immunotherapy.

Data are presented as mean±standard deviation (SD). Statistical significance was determined by Student's t-test (two-tail) between two groups, and three or more groups were analyzed by one-way analysis of variance (ANOVA). P<0.05 was considered statistically significant.

### Example 5

### GL261 Syngeneic Mouse Studies

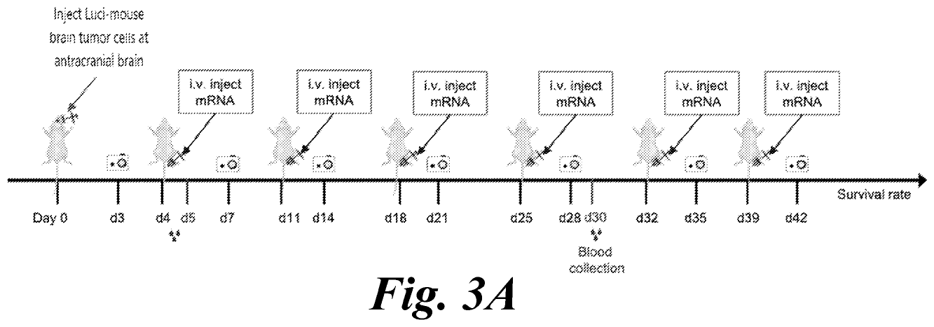

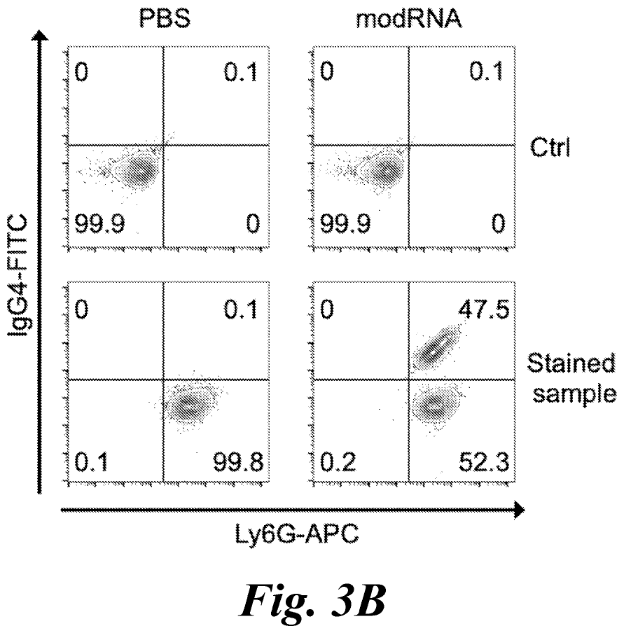

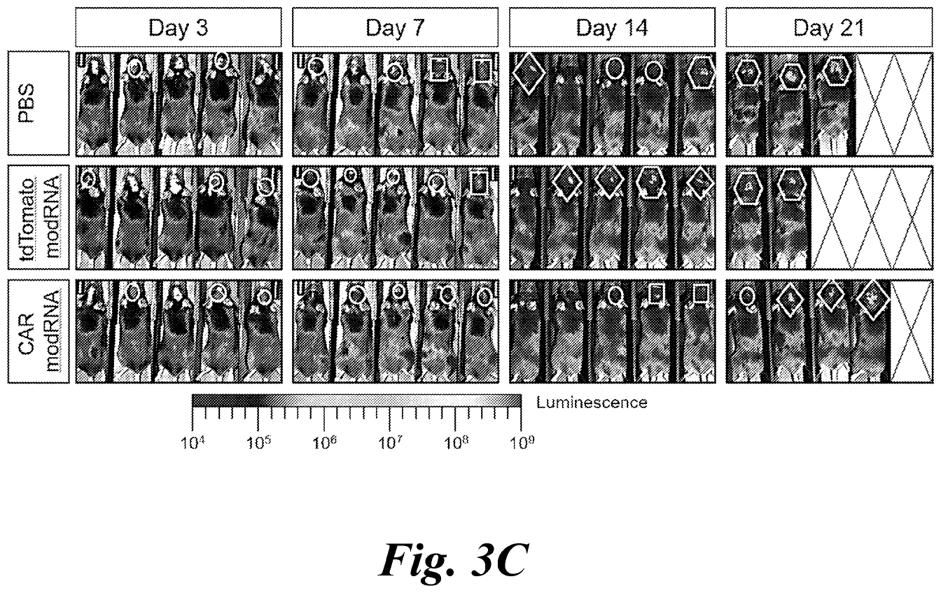

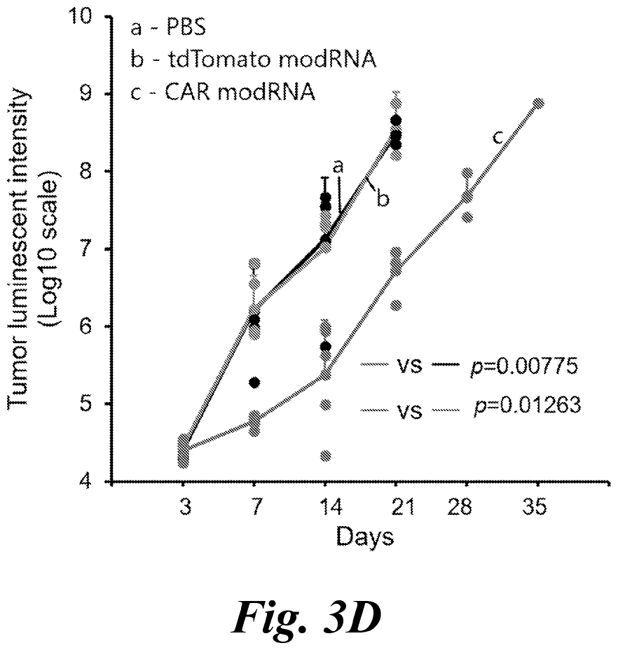

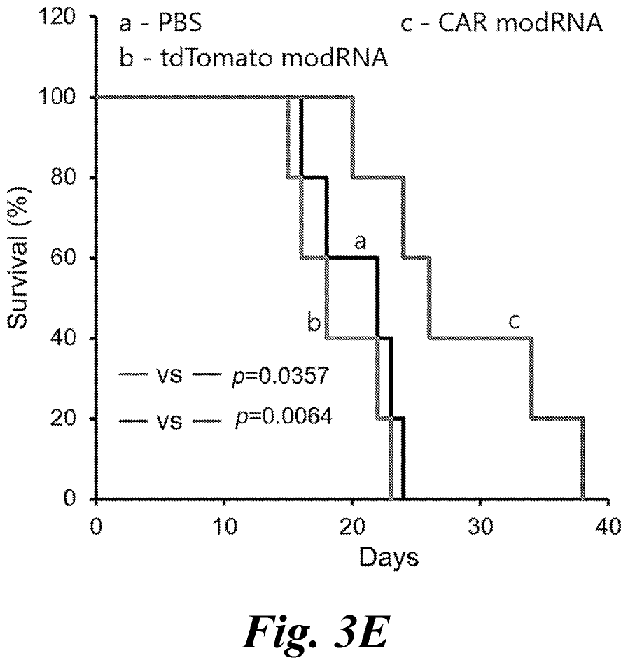

In situ syngeneic murine models were constructed via intracranial injection of 5×10⁵ luciferase-expressing-GBM GL261 cells into the brain of C57BL/6 mice. FIG. 3A shows a schematic of intravenously administered modRNA CARs and PBS for the in vivo tumor-killing study of this Example. Briefly, after tumor implantation, modRNA CARs (30 μg 5×10⁶) (the test group) or PBS (the control group) was intravenously injected into these mice at day 4, day 11, day 18, day 25, day 32, and day 39, and blood samples were collected from these mice 24 hours after modRNA administration (i.e., day 5, day 12, day 19, day 26, day 33, and day 39). Tumor burden was monitored by bioluminescence imaging (BLI) system (Spectral Ami Optical Imaging System, Spectral Instruments, Inc., Tucson, TZ), and body weights of experimental mice were measured once per week (i.e., at day 7, day 14, day 21, day 28, day 35, and day 42) (FIGS. 3C and 3D).

For in vivo biodistribution analysis, blood and immune cells were isolated from tissues or tumors and subjected to flow cytometry analysis of CAR-neutrophils 24 hours after intravenous injection of modRNA CARs. Collected blood cells were stained with IgG4-FITC and Lys6G-APC conjugated antibodies and analyzed in an Accuri C6 plus flow cytometer (Beckton Dickinson and Company, Franklin Lakes, NJ). FIG. 3B shows data from the representative flow cytometry analysis of Ly6G and CAR (IgG4) expression in the mouse peripheral blood cells measured from each group.

Blood samples were also subjected to ELISA to measure mouse cytokines, such as IL-12, IFNγ and IL-6 expression (Invitrogen, Thermo Fischer Scientific, Waltham, MA). At the end of treatment, tumors were collected for H&E staining and other histology analysis.

FIG. 3E is a Kaplan-Meier curve showing the survival of PBS or modRNA-treated groups (n=5).

### Example 6

### In Vivo Anti-Tumor Activities of modRNA CLTX-CAR Neutrophils

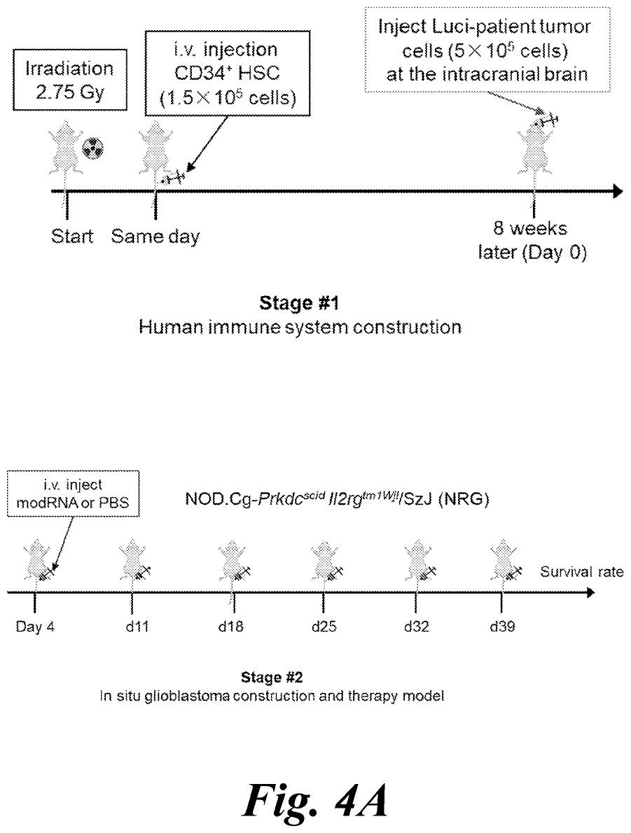

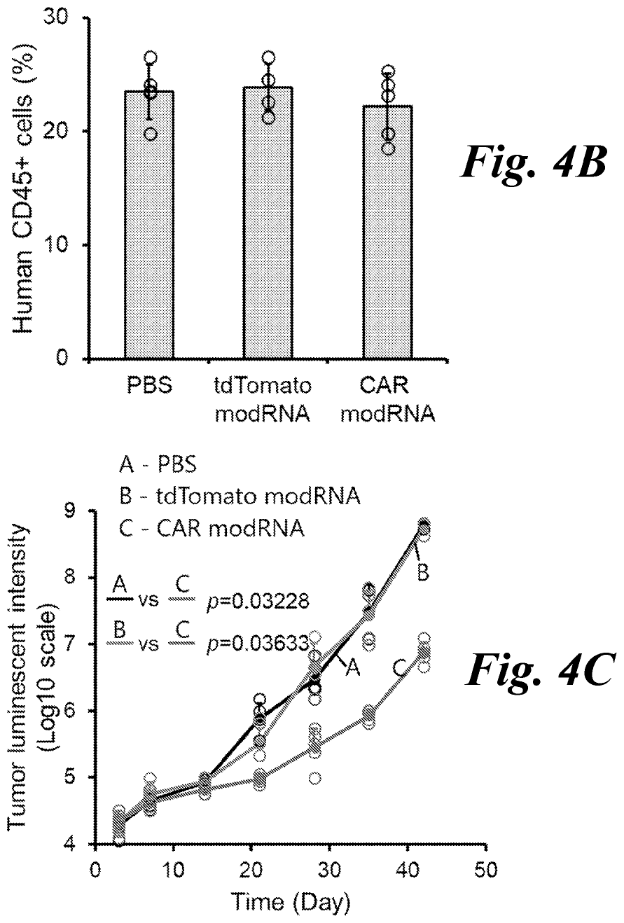

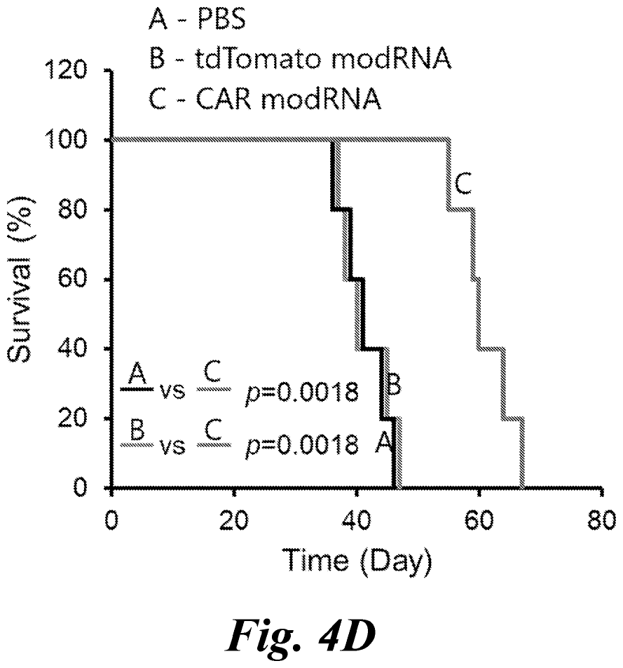

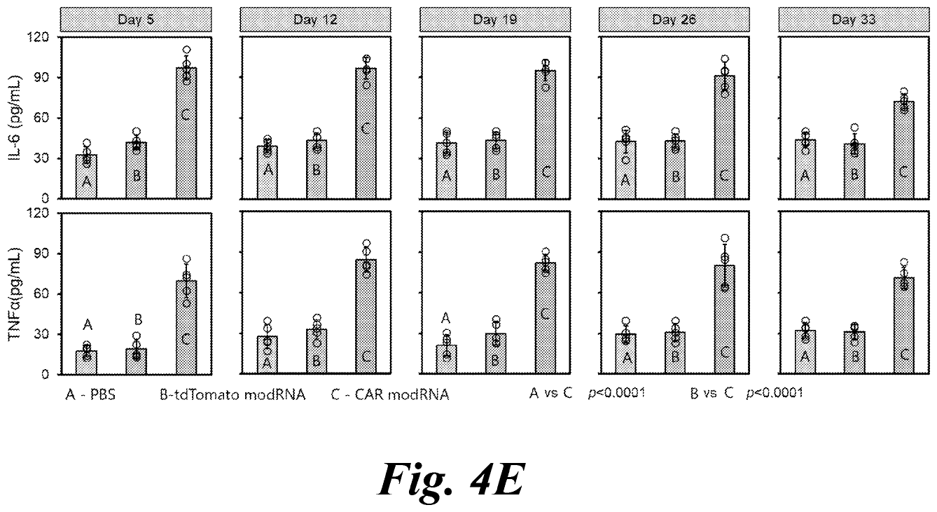

FIG. 4A shows a schematic diagram of a study that assessed the resultant neutrophil CARs' (Neu-CARs) anti-tumor activities and in vivo tumor-killing efficacy in a humanized mouse model. In stage 1 of the study, immunodeficient NOD.Cg-Prkdc^{scid} II24rg^{tm1Wjl}/SzJ (NRG) mice were exposed to irradiation at a rate of 2.75 Gy/minute and, on the same day, 1.5×10⁵ CD34⁺ HSC cells were injected (via the tail vein) into the mice. 8 weeks thereafter, 5×10⁵ luciferase (Luci)-expressing U87MG cells were injected into right forebrain of the NRG mice.

Four days after tumor implantation, tdTomato modRNA or CAR modRNA (30 μg 5×10⁶ each) (the test groups) or PBS (the control group) was intravenously injected into these humanized NRG mice weekly for six weeks (i.e., at day 4, day 11, day 18, day 25, day 32, and day 39 of the study), and blood samples were collected from these mice 24 hours after modRNA administration (i.e., day 5, day 12, day 19, day 26, day 33, and day 39). Tumor burden was monitored by BLI system (Spectral Ami Optical Imaging System, Spectral Instruments, Inc., Tucson, TZ), and body weights of experimental mice were measured once per week (i.e., at day 7, day 14, day 21, day 28, day 35, and day 42).

FIG. 4B shows quantification of human CD45⁺ cells in mouse peripheral blood measured in the different experimental groups. FIG. 4C shows quantification of the time-dependent tumor burden by BLI at the indicated days for humanized NRG mice. FIG. 4D shows a Kaplan-Meier curve demonstrating survival of the indicated experimental groups. FIG. 4E shows quantification of released human tumor necrosis factor-α (TNFα) and IL-6 in the peripheral blood of different mouse groups at the indicated days.

### Example 7

### In Vivo Anti-Tumor Activities of Combinatory modRNA CLTX-CAR Neutrophils and Chemotherapy

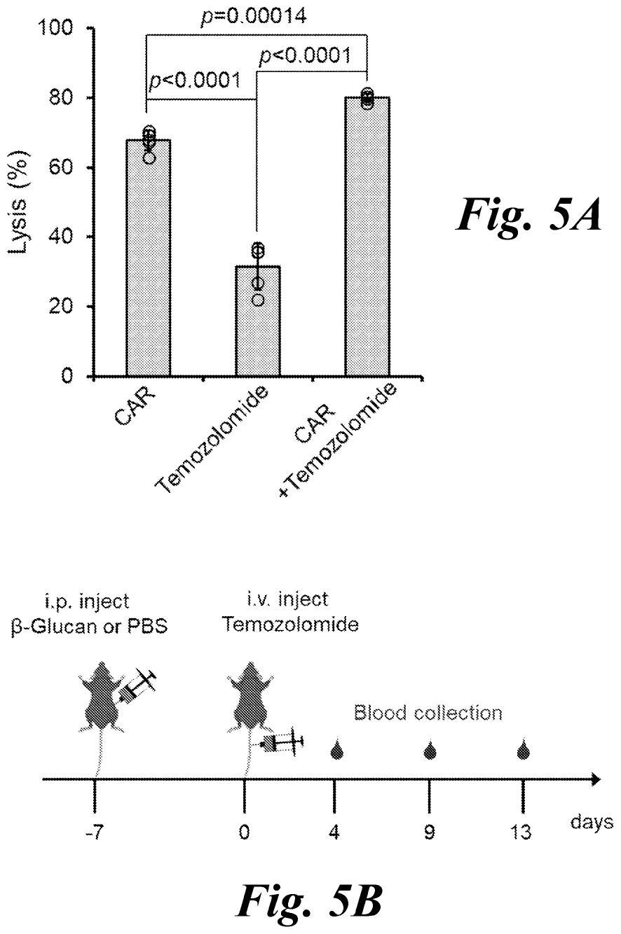

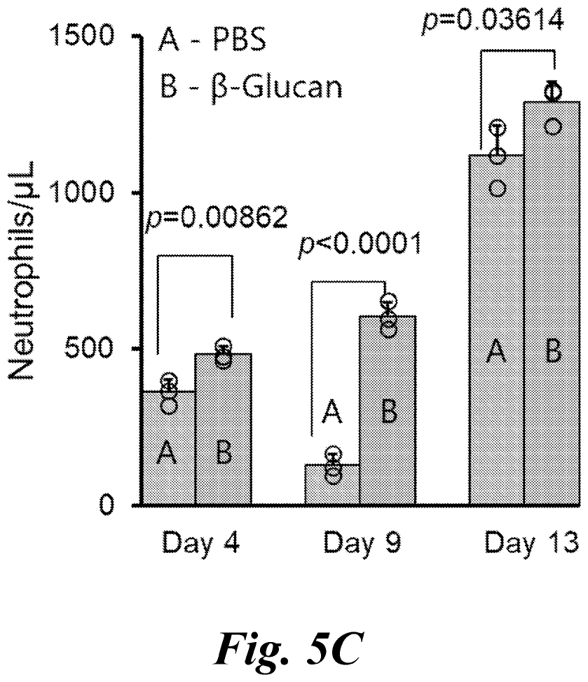

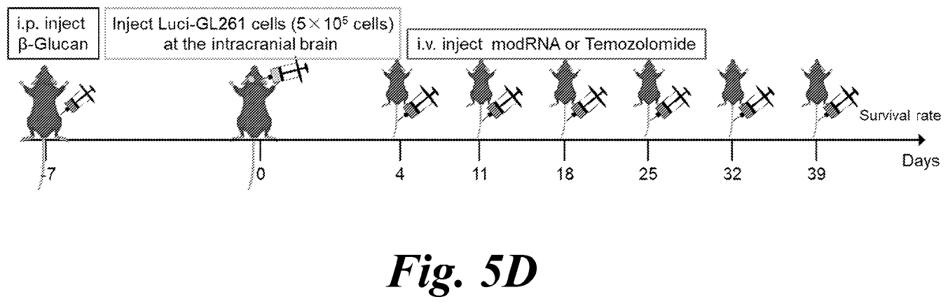

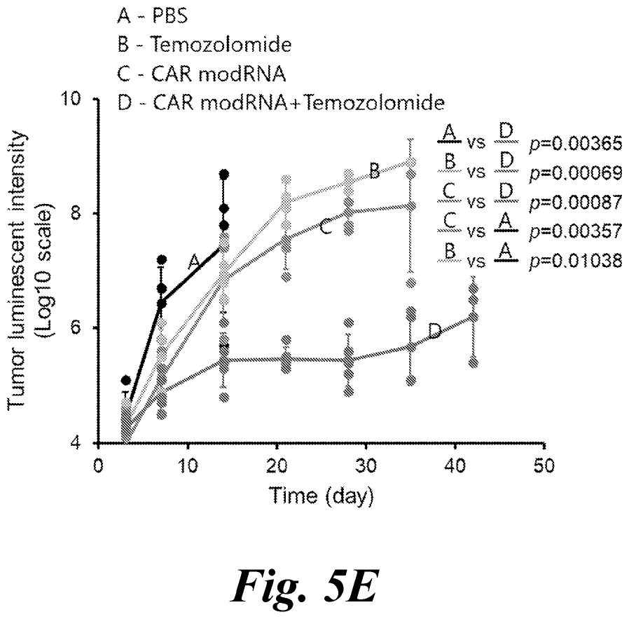

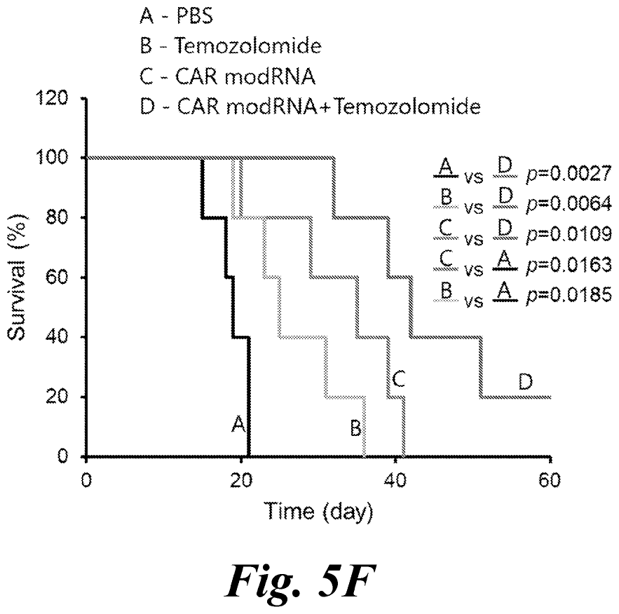

FIG. 5B shows a schematic diagram of a study that assessed anti-tumor activity and neutrophil loss following combinatory modRNA CLTX-CAR neutrophil and chemotherapy treatment. Briefly, the treatment mice were treated with β-Glucan (intraperitoneally (i.p.)) and the control group with PBS (i.p.) 7 days prior to day 0 of the study. On day 0, the chemotherapy drug Temozolomide (TMZ) was injected intravenously (through TV) to the treatment group, and blood samples were collected from these mice on days 4, 9 and 13. FIG. 5C shows the quantification of neutrophils in mouse peripheral blood taken at the indicated days.

FIG. 5D shows a schematic diagram of the study protocol for assessing the resultant combinatory therapy's anti-tumor activities and in vivo tumor-killing efficacy in the GL261 syngeneic mouse model. There, the mice were treated with β-Glucan (i.p.) 7 days prior to day 0 of the study. On day 0, 5×10⁵ Luci-expressing U87MG cells were injected into right forebrain of each group of mice. Four days thereafter, the mice were administered PBS (control group), TMZ, modRNA CARs with TMZ, or modRNA CARs alone (the latter four listed groups, the treatment groups) weekly for six weeks (i.e., at day 4, day 11, day 18, day 25, day 32, and day 39 of the study), blood samples were collected from these mice periodically, and the resultant anti-tumor activities and in vivo tumor-killing efficacy was assessed. Tumor burden was monitored by BLI system (Spectral Ami Optical Imaging System, Spectral Instruments, Inc., Tucson, TZ) (FIG. 5E), and body weights of experimental mice were measured once per week (i.e., at day 7, day 14, day 21, day 28, day 35, and day 42).

FIG. 5A shows quantification of tumor lysis against GL261 cells by modRNA CAR neutrophils, TMZ, or both. FIG. 5F shows a Kaplan-Meier curve demonstrating survival of the indicated experimental groups.

### Example 8

### Canine Studies

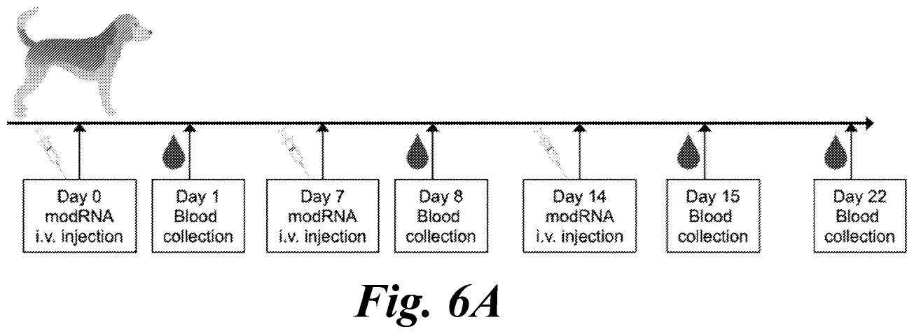

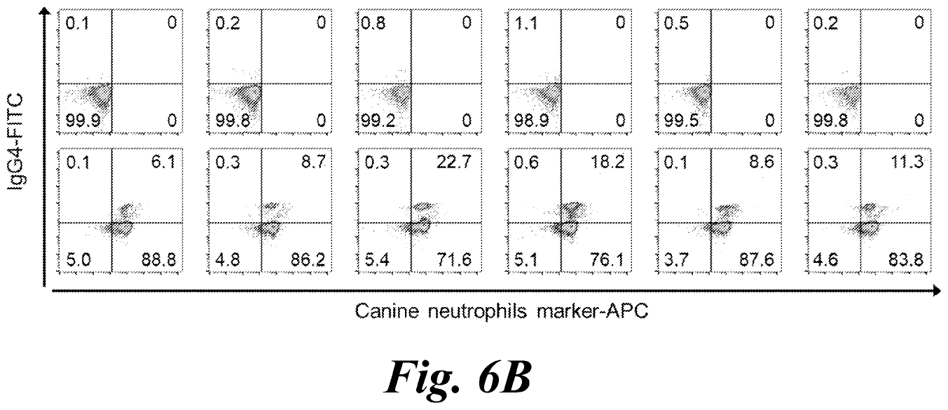

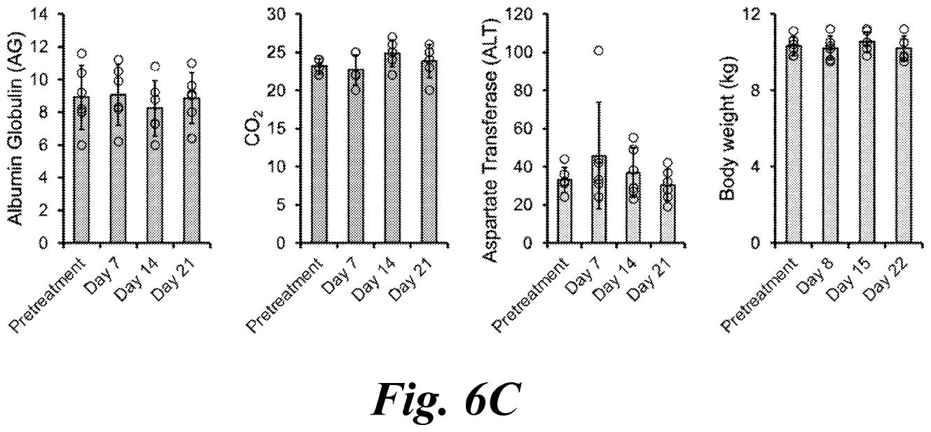

A canine model was used to assess the safety of modRNA CAR delivery to circulating neutrophils in vivo. FIG. 6A shows a schematic of intravenously administered modRNA CARs and PBS for the safety study. Briefly, modRNA CARs (30 μg 5×10⁶) (the test group) or PBS (the control group) was intravenously injected into these dogs at day 0, day 7, and day 14, and blood samples were collected at day 1, day 8, day 15 and day 22. Collected blood cells were stained with IgG4-FITC and canine neutrophil antibodies, and analyzed in an Accuri C6 plus flow cytometer (Beckton Dickinson and Company, Franklin Lakes, NJ) to measure canine neutrophil marker and IgG4 expression in the canine peripheral blood cells (FIG. 6B). Albumin globulin (AG), CO2, and aspartate transferase (ALT) levels and the body weight of each subject was measured before and after modRNA+CAR treatment (n=6) (FIG. 6C).

1. A method of producing chimeric antigen receptor (CAR)-expressing neutrophils in a subject, which method comprises administering to the subject at least one modified RNA (modRNA) construct comprising:
a first modRNA construct comprising a nucleotide sequence encoding a CAR and at least one k-turn, wherein the at least one k-turn is upstream of the nucleotide sequence encoding CAR, whereupon when the at least one modRNA construct is endocytosed by a neutrophil in the subject, and the neutrophil expresses the CAR.

2. The method of claim 1, wherein the at least one modRNA construct comprises the first modRNA construct and a second modRNA construct comprising a nucleotide sequence encoding an archaeal ribosomal protein L7Ae (L7Ae) and at least one neutrophil-specific microRNA (miRNA) recognition sequence, wherein the at least one neutrophil-specific miRNA recognition sequence is downstream of the nucleotide sequence encoding L7Ae.

3. The method of claim 2, wherein the subject is a mammal.

4. The method of claim 2, wherein the neutrophil-specific miRNA is or comprises miR-233, miR-142, or both miR-233 and miR-142.

5. The method of any one of claims 1-4, wherein the subject is a canine.

6. The method of any one of claims 1-4, wherein the subject is human.

7. The method of claim 1, wherein the CAR comprises a neutrophil-specific transmembrane domain.

8. The method of claim 7, wherein the neutrophil-specific transmembrane domain is or comprises a toll-like receptor (TLR) 4 polypeptide, a TLR2 polypeptide, a MET polypeptide, a granulocyte colony stimulating factor receptor (G-CSFR), a Myd88 polypeptide, a TRIF polypeptide, a Syk peptide, a CD40 polypeptide, CD32a, Dectin-1, a IL-6 receptor (IL6R), an Fc Epsilon Receptor Ig (FCER1G) polypeptide, a TLR7, or a CD16 transmembrane polypeptide, a CD8 polypeptide, a CD28 polypeptide, an OX40 polypeptide, an ICOS polypeptide, a CTLA-4 polypeptide, a PD-1 polypeptide, a LAG-3 polypeptide, a BTLA polypeptide, a natural killer group 2D (NKG2D), Dectin-1, CD16, or other Fcγ receptors.

9. The method of any one of claims 1, 7, and 8, wherein the CAR comprises a neutrophil-specific transmembrane and/or co-stimulatory domain, wherein:
the neutrophil-specific co-stimulatory domain can optionally be or comprise a 41BB co-stimulatory domain or a CD28 polypeptide; and
the neutrophil-specific transmembrane domain can optionally be or comprise CD32a, CD4, NKG2D, Dectin-1, a CD3 polypeptide, an IL-6 receptor, or CD16.

10. The method of any one of claims 1-4, 7, and 8, wherein the subject is human and the CAR comprises a human CD4 transmembrane domain and a human CD3ζ intracellular domain.

11. The method of any one of claims 1-4, 7, and 8, wherein the subject is human and the CAR comprises a human CD8 transmembrane domain, a human 41BB co-stimulatory domain, and a human CD3ζ intracellular domain.

12. The method of claim any one of claims 1-4, 7, and 8, wherein the CAR comprises a 36-amino acid glioblastoma (GBM)-targeting chlorotoxin peptide or other brain-targeting ligand, a tumor-targeting ligand, or a single-chain variable fragment (scFv).

13. The method of any one of claims 1-4, 7, and 8, wherein the CAR comprises a ligand that targets prostate-specific membrane antigen (PSMA).

14. The method of claim 1 or 2, wherein the modRNA construct(s) is/are administered in an exosome to circulating neutrophils in vivo.

15. The method of claim 1 or 2, wherein the modRNA construct(s) is/are administered systemically to the subject in an exosome to circulating neutrophils in vivo.

16. The method of claim 1 or 2, wherein the modRNA construct(s) is/are administered in a lipid nanoparticle (LNP).

17. The method of claim 1 or 2, wherein the modRNA construct(s) is/are administered systemically to the subject in a LNP.

18. The method of any one of claims 1-4, 7, and 8, further comprising administering a therapeutically effective amount of a drug, optionally a nanodrug and/or a prodrug, to the subject.

19. The method of any one of claims 1-4, 7, and 8, further comprising administering a second therapy to the subject.

20. The method of any one of claims 1-4, 7, and 8, further comprising administering a second therapy to the subject, wherein the second therapy comprises surgical removal of one or more cancerous cells from the subject, chemotherapy, imaging, and/or radiotherapy.

21. The method of any one of claims 1, 2, and 7-10, further comprising administering a therapeutically effective amount of a nanodrug to the subject, wherein the nanodrug is a chemotherapeutic agent.

22. The method of any one of claims 1-21, wherein the subject has cancer.

23. The method of claim 1 or 2, wherein the subject has brain cancer.

24. The method of claim 1 or 2, wherein the brain cancer is glioblastoma.

25. The method of claim 1 or 2, wherein the subject has prostate cancer.

26. The method of claim 1, wherein the neutrophil that endocytosis the at least one modRNA construct is an isolated neutrophil and the method further comprises administering the isolated neutrophil that endocytosed the at least one modRNA construct to the subject.

27. The method of claim 26, further comprising isolating one or more neutrophils from the subject.

28. The method of any one of claim 2-4, 7, 8, 26, or 27, wherein the at least one neutrophil-specific microRNA recognition sequence of the second modRNA construct is or comprises SEQ ID NO: 3, SEQ ID NO: 4, SEQ ID NO: 5, SEQ ID NO: 6, SEQ ID NO: 7, or a functional variant of SEQ ID NO: 3, SEQ ID NO: 4, SEQ ID NO: 5, SEQ ID NO: 6, or SEQ ID NO: 7.

29. The method of any one of claim 1-4, 7, 8, 26, or 27, wherein the first modRNA construct comprises SEQ ID NO: 8, SEQ ID NO: 9, or a functional variant of SEQ ID NO: 8 or SEQ ID NO: 9.

30. The method of any one of claim 1-4, 7, 8, 26, or 27, wherein the first modRNA construct comprises SEQ ID NO: 10, SEQ ID NO: 11, SEQ ID NO: 12, or a functional variant of SEQ ID NO: 10, SEQ ID NO: 11, or SEQ ID NO: 12.

31. The method of any one of claim 1-4, 7, 8, 26, or 27, wherein the first modRNA construct comprises SEQ ID NO: 1, SEQ ID NO: 2, or a functional variant of SEQ ID NO: 1 or SEQ ID NO: 2.

32. The method of any one of claim 1-4, 7, 8, 26, or 27, wherein the first modRNA construct comprises:
a first sequence comprising SEQ ID NO: 1, SEQ ID NO: 2, or a functional variant of SEQ ID NO: 1 or SEQ ID NO: 2; and
a second sequence comprising SEQ ID NO: 10, SEQ ID NO: 11, SEQ ID NO: 12, or a functional variant of SEQ ID NO: 10, SEQ ID NO: 11, or SEQ ID NO: 12;
wherein the first sequence is upstream of the second sequence.

33. A method of producing chimeric antigen receptor (CAR)-expressing neutrophils ex vivo for administration to a subject, which method comprises:
(i) isolating neutrophils from a subject,
(ii) contacting the isolated neutrophils with at least one modified RNA (modRNA) construct, the at least one modRNA construct comprising a first modRNA construct comprising a nucleotide sequence encoding a CAR and at least one k-turn, wherein the at least one k-turn is upstream of the nucleotide sequence encoding CAR;
whereupon when the modRNA is endocytosed by the isolated neutrophils, the isolated neutrophils express the CAR, and
(iii) administering the CAR-expressing neutrophils to the subject.

34. The method of claim 33, wherein the at least one modRNA construct comprises the first modRNA construct and a second modRNA construct, the second modRNA construct comprising a nucleotide sequence encoding an archaeal ribosomal protein L7Ae (L7Ae) and at least one neutrophil-specific microRNA (miRNA) recognition sequence, wherein the at least one neutrophil-specific miRNA recognition sequence is downstream of the nucleotide sequence encoding L7Ae.

35. The method of claim 34, wherein the subject is human and the at least one neutrophil-specific miRNA recognition sequence of the second modRNA construct is or comprises miR-233, miR-142, or both miR-233 and miR-142.

36. The method of any one of claims 33-35, wherein the subject is human and the CAR comprises a human CD4 transmembrane domain and a human CD3ζ intracellular domain.

37. The method of any one of claims 33-35, wherein the subject is human and the CAR comprises a human CD8 transmembrane domain, a human 41BB co-stimulatory domain, and a human CD3ζ intracellular domain.

38. The method of any one of claims 33-35, wherein the CAR comprises a 36-amino acid glioblastoma (GBM)-targeting chlorotoxin peptide or other brain tumor-targeting ligand.

39. The method of any one of claims 33-35, wherein the CAR comprises a ligand that targets prostate-specific membrane antigen (PSMA).

40. The method of any one of claims 33-35, wherein the CAR comprises a neutrophil-specific transmembrane domain.

41. The method of any one of claims 33-35, wherein the CAR comprises a neutrophil-specific transmembrane domain is or comprises a toll-like receptor (TLR) 4 polypeptide, a TLR2 polypeptide, a MET polypeptide, a granulocyte colony stimulating factor receptor (G-CSFR), a Myd88 polypeptide, a TRIF polypeptide, a Syk peptide, a CD40 polypeptide, CD32a, Dectin-1, a IL-6 receptor (IL6R), an Fc Epsilon Receptor Ig (FCER1G) polypeptide, a TLR7, or a CD16 transmembrane polypeptide, a CD8 polypeptide, a CD28 polypeptide, an OX40 polypeptide, an ICOS polypeptide, a CTLA-4 polypeptide, a PD-1 polypeptide, a LAG-3 polypeptide, a BTLA polypeptide, a natural killer group 2D (NKG2D), Dectin-1, CD16, or other Fcγ receptors.

42. The method of any one of claims 33-35, wherein the CAR comprises a neutrophil-specific transmembrane and/or co-stimulatory domain, wherein:
the neutrophil-specific co-stimulatory domain can optionally be or comprise a 41BB co-stimulatory domain or a CD28 polypeptide; and
the neutrophil-specific transmembrane domain can optionally be or comprise CD32a, CD4, NKG2D, Dectin-1, a CD3 polypeptide, an IL-6 receptor, or CD16.

43. The method of any one of claims 33-35, which further comprises administering to the subject a therapeutically effective amount of a drug and, optionally, the drug comprises a nanodrug or a prodrug.

44. The method of any one of claims 33-35, which further comprises administering to the subject a therapeutically effective amount of a chemotherapeutic agent.

45. The method of any one of claims 33-35, wherein the subject has cancer.

46. The method of any one of claims 33-35, wherein the subject has a brain cancer.

47. The method of any one of claims 33-35, wherein the subject has glioblastoma.

48. The method of any one of claims 33-35, wherein the subject has prostate cancer.

49. The method of claim 34 or 35, wherein the at least one neutrophil-specific microRNA recognition sequence of the second modRNA construct is or comprises SEQ ID NO: 3, SEQ ID NO: 4, SEQ ID NO: 5, SEQ ID NO: 6, SEQ ID NO: 7, or a functional variant of SEQ ID NO: 3, SEQ ID NO: 4, SEQ ID NO: 5, SEQ ID NO: 6, or SEQ ID NO: 7.

50. The method of any one of claims 33-35, wherein the first modRNA construct comprises SEQ ID NO: 8, SEQ ID NO: 9, or a functional variant of SEQ ID NO: 8 or SEQ ID NO: 9.

51. The method of any one of claims 33-35, wherein the first modRNA construct comprises SEQ ID NO: 10, SEQ ID NO: 11, SEQ ID NO: 12, or a functional variant of SEQ ID NO: 10, SEQ ID NO: 11, or SEQ ID NO: 12.

52. The method of any one of claims 33-35, wherein the first modRNA construct comprises SEQ ID NO: 1, SEQ ID NO: 2, or a functional variant of SEQ ID NO: 1 or SEQ ID NO: 2.

53. The method of any one of claims 33-35, wherein the first modRNA construct comprises:
a first sequence comprising SEQ ID NO: 1, SEQ ID NO: 2, or a functional variant of SEQ ID NO: 1 or SEQ ID NO: 2; and
a second sequence comprising SEQ ID NO: 10, SEQ ID NO: 11, SEQ ID NO: 12, or a functional variant of SEQ ID NO: 10, SEQ ID NO: 11, or SEQ ID NO: 12;
wherein the first sequence is upstream of the second sequence.

54. A composition comprising a first modRNA construct comprising a nucleotide sequence encoding a chimeric antigen receptor (CAR) and at least one k-turn, wherein the at least one k-turn is upstream of the nucleotide sequence encoding the CAR.

55. The composition of claim 54 further comprising a second modRNA construct comprising a nucleotide sequence encoding the archaeal ribosomal protein L7Ae and at least one neutrophil-specific microRNA (miRNA) recognition sequence, wherein the at least one neutrophil-specific miRNA recognition sequence is downstream of the nucleotide sequence encoding L7Ae.

56. The composition of claim 55, wherein the at least one neutrophil-specific miRNA recognition sequence of the second modRNA construct is or comprises miR-233, miR-142, or both miR-233 and miR-142.

57. The composition of any one of claims 54-56, wherein the CAR comprises a human CD4 transmembrane domain and a human CD3ζ intracellular domain.

58. The composition of any one of claims 54-56, wherein the CAR comprises a human CD8 transmembrane domain, a human 41BB co-stimulatory domain, and a human CD3ζ intracellular domain.

59. The composition of any one of claims 54-56, wherein the CAR comprises a 36-amino acid glioblastoma (GBM)-targeting chlorotoxin peptide or other brain tumor-targeting ligand (e.g., IL-13) or a single-chain variable fragment (scFv).

60. The composition of any one of claims 54-56, wherein the CAR comprises a ligand that targets prostate-specific membrane antigen (PSMA).

61. The composition of claim 55 or 56, wherein the at least one neutrophil-specific miRNA recognition sequence of the second modRNA construct is or comprises SEQ ID NO: 3, SEQ ID NO: 4, SEQ ID NO: 5, SEQ ID NO: 6, SEQ ID NO: 7, or a functional variant of SEQ ID NO: 3, SEQ ID NO: 4, SEQ ID NO: 5, SEQ ID NO: 6, or SEQ ID NO: 7.

62. The composition of any one of claims 54-56, wherein the first modRNA construct comprises SEQ ID NO: 8, SEQ ID NO: 9, or a functional variant of SEQ ID NO: 8 or SEQ ID NO: 9.

63. The composition of any one of claims 54-56, wherein the first modRNA construct comprises SEQ ID NO: 10, SEQ ID NO: 11, SEQ ID NO: 12, or a functional variant of SEQ ID NO: 10, SEQ ID NO: 11, or SEQ ID NO: 12.

64. The composition of any one of claims 54-56, wherein the first modRNA construct comprises SEQ ID NO: 1, SEQ ID NO: 2, or a functional variant of SEQ ID NO: 1 or SEQ ID NO: 2.

65. The composition of any one of claims 54-56, wherein the first modRNA construct comprises:
a first sequence comprising SEQ ID NO: 1, SEQ ID NO: 2, or a functional variant of SEQ ID NO: 1 or SEQ ID NO: 2; and
a second sequence comprising SEQ ID NO: 10, SEQ ID NO: 11, SEQ ID NO: 12, or a functional variant of SEQ ID NO: 10, SEQ ID NO: 11, or SEQ ID NO: 12;
wherein the first sequence is upstream of the second sequence.

66. The composition of any one of claims 54-56, wherein the CAR comprises a neutrophil-specific transmembrane domain.

67. The composition of any one of claims 54-56, wherein the CAR comprises a neutrophil-specific transmembrane domain that is or comprises a toll-like receptor (TLR) 4 polypeptide, a TLR2 polypeptide, a MET polypeptide, a granulocyte colony stimulating factor receptor (G-CSFR), a Myd88 polypeptide, a TRIF polypeptide, a Syk peptide, a CD40 polypeptide, CD32a, Dectin-1, a IL-6 receptor (IL6R), an Fc Epsilon Receptor Ig (FCER1G) polypeptide, a TLR7, or a CD16 transmembrane polypeptide, a CD8 polypeptide, a CD28 polypeptide, an OX40 polypeptide, an ICOS polypeptide, a CTLA-4 polypeptide, a PD-1 polypeptide, a LAG-3 polypeptide, a BTLA polypeptide, a natural killer group 2D (NKG2D), Dectin-1, CD16, or other Fcγ receptors.

68. The composition of any one of claims 54-56, wherein the CAR comprises a neutrophil-specific transmembrane and/or co-stimulatory domain, wherein:
the neutrophil-specific co-stimulatory domain can optionally be or comprise a 41BB co-stimulatory domain or a CD28 polypeptide; and
the neutrophil-specific transmembrane domain can optionally be or comprise CD32a, CD4, NKG2D, Dectin-1, a CD32 polypeptide, an IL-6 receptor, or CD16.

69. An exosome comprising the composition of any one of claims 54-68.

70. A nanoparticle comprising the composition of any one of claims 54-68.

71. The nanoparticle of claim 70, comprising a lipid nanoparticle.

72. Use of at least one modified RNA (modRNA) constructs in the preparation of a medicament for treating a cancer, the at least one modRNA construct comprising:
a first modRNA construct comprising a nucleotide sequence encoding a CAR and at least one k-turn.

73. The use of claim 72, wherein the at least one modRNA constructs comprises a combination of the first modRNA construct and a second modRNA construct, the second modRNA construct comprising a nucleotide sequence encoding an archaeal ribosomal protein L7Ae (L7Ae) and at least one neutrophil-specific microRNA recognition sequence, wherein the at least one neutrophil-specific microRNA recognition sequence is downstream of the nucleotide sequence encoding L7Ae.

74. The use of claim 72 or 73, wherein the cancer is a brain cancer.

75. The use of claim 72 or 73, wherein the cancer is glioblastoma.

76. The use of claim 72 or 73, wherein the cancer is a prostate cancer.

77. The use of claim 73, wherein the second modRNA construct comprises at least one neutrophil-specific microRNA recognition sequence that is or comprises miR-233, miR-142, or both miR-233 and miR-142.

78. The use of any one of claims 72-77, wherein the CAR comprises a human CD4 transmembrane domain and a human CD3ζ intracellular domain.

79. The use of any one of claims 72-77, wherein the CAR comprises a human CD8 transmembrane domain, a human 41BB co-stimulatory domain, and a human CD3ζ intracellular domain.

80. The use of any one of claims 72-77, wherein the CAR comprises a 36-amino acid glioblastoma (GBM)-targeting chlorotoxin peptide or other brain tumor-targeting ligand or a single-chain variable fragment (scFv).

81. The use of any one of claims 72-77, wherein the CAR comprises a ligand that targets prostate-specific membrane antigen (PSMA).

82. The use of claim 72, wherein the at least one modRNA constructs are encapsulated within a carrier.

83. The use of claim 82 or 83, wherein the medicament is formulated for systemic administration.

84. The use of claim 82 or 83, wherein the at least one modRNA constructs are encapsulated within a nanoparticle (NP), a microvesicle, or an exosome.

85. The use of claim 82 or 83, wherein the medicament is formulated for administration in combination with a therapeutically effective amount of a drug, optionally a nanodrug.


SEQUENCE LISTING (14 sequences)

SEQ ID NO: 1 - DNA, 23 bp, synthetic construct
  source 1..23: mol_type = other DNA; note = encodes a k-turn
gggcgtgatccgaaaggtgaccc

SEQ ID NO: 2 - DNA, 23 bp, synthetic construct
  source 1..23: mol_type = other DNA; note = encodes a k-turn
gggcgtgatgcgaaagctgaccc

SEQ ID NO: 3 - DNA, 22 bp, synthetic construct
  source 1..22: mol_type = other DNA; note = encodes a microRNA233 binding site
tggggtatttgacaaactgaca

SEQ ID NO: 4 - DNA, 21 bp, synthetic construct
  source 1..21: mol_type = other DNA; note = encodes a microRNA-223 binding site with a bulge
tggggtatttctgaactgaca

SEQ ID NO: 5 - DNA, 23 bp, synthetic construct
  source 1..23: mol_type = other DNA; note = encodes a microRNA-142-5p binding site
agtagtgctttctactttatggg

SEQ ID NO: 6 - DNA, 22 bp, synthetic construct
  source 1..22: mol_type = other DNA; note = encodes a miRNA-142-5p binding site with a bulge-1
agtagtgctttggatttatggg

SEQ ID NO: 7 - DNA, 22 bp, synthetic construct
  source 1..22: mol_type = other DNA; note = encodes a miRNA-142-5p binding site with a bulge-2
agtagtgctagaactttatggg

SEQ ID NO: 8 - protein, 387 aa, synthetic construct
  source 1..387: note = protein sequence for neutrophil-specific L7Ae:k-turn RNP switch
MYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKPKKKRKV

SEQ ID NO: 9 - protein, 476 aa, synthetic construct
  source 1..476: note = modRNA tdTomato construct comprising two k-turns recognized by L7Ae
MVSKGEEVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLGHGTGSTGSGSSGTASSEDNNMAVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLYGMDELYK

SEQ ID NO: 10 - protein, 474 aa, synthetic construct
  source 1..474: note = protein sequence for 2x k-turn CLTX human 4-1BB CD3z chimeric antigen receptor
MLLLVTSLLLCELPHPAFLLIPMCMPCFTTDHQMARKCDDCCGGKGRGKCYGPQCLCRESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 468 aa, synthetic construct
  source 1..468: note = protein sequence for 2x k-turn CLTX mouse CD28 CD3z CAR
MLLLVTSLLLCELPHPAFLLIPMCMPCFTTDHQMARKCDDCCGGKGRGKCYGPQCLCRESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWALVVVAGVLFCYGLLVTVALCVIWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 12 - protein, 470 aa, synthetic construct
  source 1..470: note = protein sequence for a CLTX canine CD28 CD3z CAR
MLLLVTSLLLCELPHPAFLLIPMCMPCFTTDHQMARKCDDCCGGKGRGKCYGPQCLCRESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWALVVVGAVLVFYSLLVTVALCAYWIKSKSSRILQSDYMNMTPRRPGPTRRHYQPYAPARDFAAYRSLRAKFGRSAAAPEHQQGPNQLYNELNLRGREEYEVLDKRRGLDPEMGGKQRKRNPQEVVYNALQKDKMAEAYSEIGIKSENQRRRGKGHDGLYQGLSTATKDTYDALHMQALPPR

SEQ ID NO: 13 - protein, 10 aa, synthetic construct
  source 1..10: note = peptide that targets mouse neutrophils
LQIQSWSSSP

SEQ ID NO: 14 - protein, 15 aa, synthetic construct
  source 1..15: note = peptide that targets human neutrophils
KFPDLDSRRLPHMSL